(19) United States
(12) Patent Application Publication
(10) Pub. No.: US 2007/0167775 A1
(43) Pub. Date: Jul. 19, 2007

(54) Method and apparatus for protecting the rectal wall during cryoablation

(75) Inventors: Eyal Kochavi, Haifa (IL); Amir Pansky, Atlit (IL); Yaron Hefetz, Herzlia (IL)

Correspondence Address: Martin D. Moynihan;PRTSI, Inc., P.O. Box 16446, Arlington, VA, 22215, US

(73) Assignee: Galil Medical Ltd., Yokneam (IL)

(21) Appl. No.: 11/638,347

(22) Filed: Dec. 14, 2006

Related U.S. Application Data
(60) Provisional application No. 60/750,344, filed on Dec. 15, 2005.

(51) Int. Cl. A61B 8/00

(52) U.S. Cl. 600/439

(57) ABSTRACT

The present invention relates to apparatus and methods for protecting walls of a body cavity during thermal ablation of tissues near that body cavity, while enabling ultrasound monitoring of the ablation process. Heating protective devices and cooling protective devices are provided. Heatable/coolable ultrasound probes and heatable/coolable independent protective devices compatible with ultrasound imaging by moving ultrasound probes are provided.

### RELATED APPLICATIONS

This Application is being filed concurrently with U.S. patent application titled “Method and Apparatus For Protecting the Rectal Wall During Cryoablation” (having Attorney Docket No. 33188), and also claims the benefit of U.S. Provisional Patent Application No. 60/750,344 filed Dec. 15, 2005, the contents of which is incorporated herein by reference.

### FIELD AND BACKGROUND OF THE INVENTION

The present invention relates to an apparatus and method for protecting the wall of a body cavity during thermal ablation of tissues near that body cavity, and in particular to warming the rectal wall adjacent to the prostate during prostatic cryoablation to minimize the risk of freezing and damage to the rectum.

During prostate cryotherapy, there is a need to provide efficient full prostate ablation while keeping other organs intact, in particular the urethra, neurovascular Bundle (NVB) and rectum wall.

The general need for accurately delimiting cryoablation, that is, for limiting tissue destruction (insofar as possible) to tissues which are the intended ablation target, is discussed in U.S. application No. 20050143723 entitled “Method for delimiting cryoablation by controlled cooling”, by Zvuloni, Roni; et al., filed on Jun. 30, 2005. This application discloses a systems and methods for planning and for facilitating a cryoablation procedure. Methods not involving heating are there provided for using a plurality of cryoprobes to generate a cold field of tailored size, shape, and intensity, for cryoablating a volume with sharply defined borders, thereby minimizing damage to healthy tissues adjacent to a cryoablation target.

It is well known in the art to warm the urethra during cryotherapy of the prostate, by circulating warm water in closed loop catheter to keep the urethra above freezing temperature. U.S. Pat. No. 6,419,690 entitled “Urethral warming catheter”, to Mikus; Paul W. and Crockett; K. David, published on Jul. 16, 2002, discloses a warming catheter for use during cryosurgical ablation of the prostate having a warming section with diffuser ports promoting even warming of the prostatic urethra; the warming catheter is also provided with a short monorail lumen, monorail tip lumen, or a full length monorail lumen.

Use of a rectal ultrasound probe is common practice during cryoablation of the prostate, the rectal probe being used to monitor positioning of ablation needles in the prostate and to monitor and help control the size and position of the iceball(s) created by the ablation process. It is thus important that any processes used to protect the rectal wall during cryoablation for the prostate not interfere with processes of ultrasound imaging by means of rectal ultrasound probe.

A paper entitled “Feasibility and toxicity of transrectal ultrasound hyperthermia in the treatment of locally advanced adenocarcinoma of the prostate” by Fosmire, H., Hynynen, K., Drach, G. W., Stea, B., Swift, P., And Cassady, J. R., published in International Journal of Radiation Oncology, Biology, Physics, 26, 1993 pp 253-259, discloses a method of protecting the rectal wall during heat ablation of prostate tissues. The method there presented involves cooling the rectal wall using water circulation during treatment of the prostate by high-intensity focused ultrasound. waves, which treatment heats prostate tissues-and tends to inappropriately heat the rectal wall as well.

It is a purpose of the present invention to provide devices and methods for protecting the rectal wall during cryoablation of a prostate. The device are designed for use with an ultrasound probe, and provides means for protecting the rectum by heating the rectal wall during cryogenic cooling of tissues adjacent to the wall, while yet enabling uninterrupted and undisturbed ultrasonic imaging of the prostate and of the cryosurgical process therein by means of a rectal ultrasound probe inserted in the rectum and active during protective warming of the rectal wall.

U.S. Pat. No. 6,932,771B2 to Whitmore et al. presents systems designed to provide for both rectal heating and rectal ultrasound probe use. These applications teach use of pump and fluid (e.g. liquid) pumped through an inflatable sheath designed to hold an ultrasound probe, and heating of said circulating liquid to heat a rectum.

However, Whitmore's system presents a variety of disadvantages. Use of pumps in an open fluid circulation system adds a level of complexity and inconvenience to the cryosurgery process, particularly since liquids introduced into the body during a surgical procedure must of necessity be sterile and completely non-toxic and biocompatible. Thus there is a widely felt need for, and it would be highly advantageous to have, a tissue protection device absent the disadvantages of an open fluid circulatory system.

More importantly, Whitmore's systems as designed and as described comprise sleeves and sheaths which are immovable with respect to the ultrasonic probes when those probes are in use. Whitmore's sheaths are designed to be fixedly positioned with respect to the ultrasonic probes. Movement of Whitmore's probes therefore implies movement of his sheath as well. This, however, constitutes a serious disadvantage of his system, because in practice surgeons often find it desirable or necessary to move their ultrasonic probes forward and backward within the rectum during the cryoablation process, so as to successfully observe various aspects of body anatomy, cryoprobe placement and iceball growth. Yet, when the ultrasound probe of Whitmore's invention is moved within the rectum, the heating mechanism he provides necessarily moves along with it. Movement of the heating mechanism along with the ultrasound probe is extremely disadvantageous, in that it results in leaving portions of the rectum unprotected by the heating mechanism during periods of active cryoablation, and thereby risks inappropriate freezing of the rectum, with resultant danger of rectal fistulas or other similar forms of damage to the would-be protected tissues. Even should a surgeon manage to move Whitmore's ultrasound probe and heating sheath rapidly and briefly to accomplish ultrasound viewing while avoiding damage, the necessity for manipulating the ultrasound probe in such an inconvenient manner clearly adds to the danger and the complexity of the cryosurgical procedure.

Thus, there is a widely felt need for, and it would be highly advantageous to have, a device for protecting the rectum from cold damage, which device enables ultrasound monitoring of freezing during cooling by cryoprobes, protects the rectal wall from freezing during this procedure, and, importantly, enables movement of an ultrasound probe backwards and forwards within the rectum during the procedure. It is important that the warming mechanism be physically compatible with the form and function of the ultrasound probe, yet also that it be physically distinct from it and not fixedly connected to it, enabling movement of the ultrasound probe within the rectum to be unconstrained during all phases of the cryosurgery procedure. Indeed, it is highly desirable to have an ultrasound-compatible heating device which not only enables to advance and retract the ultrasound probe within the rectum during cryoablation, but which also enables complete removal of the ultrasound probe from the rectum during cryoablation while yet leaving the heating mechanism in place for protection of tissues.

Notwithstanding the above, for purposes of simplicity of operation, it would also be highly desirable to have an ultrasound probe which is in itself capable of supplying heat to its immediate environment, so as to protect adjacent tissues during cryoablation of a nearby ablation target while functioning as an instrument of ultrasonic visualization. It is thus a further objective of the present invention to provide such a probe.

### SUMMARY OF THE INVENTION

According to one aspect of the present invention there is provided a device for facilitating ablation of tissues near a body cavity, comprising a flexible thermal conditioner insertable together with an ultrasound probe into the body cavity, wherein at least a portion of the thermal conditioner comprises ultrasound-transmissive material operable to transmit ultrasound waves between the ultrasound probe and body tissues external to the body cavity when the thermal conditioner and the ultrasound probe are together inserted into the body cavity and the ultrasound probe is operated to image tissues, and the thermal conditioner is so shaped and surfaced as to allow the ultrasound probe to move freely relative to the conditioner when the conditioner and the ultrasound probe are together inserted in the body cavity.

According to further features in preferred embodiments of the invention described below, the thermal conditioner is formed as a sleeve having a lumen sized to accommodate an ultrasound probe, or is shaped as a pouch having a distal pocket sized to accommodate a distal end of an ultrasound probe. Preferably, the thermal conditioner further comprises electrical a conductive rubber electrical resistance heater and an immobilizer operable to immobilize the thermal conditioner with respect to the body cavity when the thermal conditioner is inserted in the body cavity.

According to another aspect of the present invention there is provided a device for facilitating ablation of tissues near a body cavity, comprising a flexible sleeve insertable in the body cavity, the device comprises an internal lumen sized to accommodate an ultrasound probe inserted in the sleeve, wherein at least a portion of the sleeve comprises ultrasound-transmissive material operable to transmit ultrasound energy between an ultrasound probe inserted in the sleeve and body tissues external to sleeve; and the internal lumen is so sized and surfaced as to allow an ultrasound probe inserted therein to move freely within the sleeve when the sleeve is inserted in the body cavity. Preferably, the sleeve is so constructed as to maintain thermal contact with a rectal wall when the sleeve is inserted in a rectum and an ultrasonic probe initially inserted in the sleeve is withdrawn therefrom.

The device preferably comprises a thermal conditioning element which is a heating element or a cooling element. The heating element may be an electrical resistance heater such as a strip of conductive rubber operable to heat when traversed by an electric current. Alternatively, the electrical resistance heater comprises electrical resistance wires encased in a flexible, ultrasound-transparent material.

Alternatively, the device comprises a conduit operable to conduct a fluid through at least a portion of the sleeve and the device comprises a pump for pumping a fluid through the conduit. The pump is preferably a peristaltic pump and the conduit is formed as a closed-circuit conduit.

According to further features in preferred embodiments of the invention described below the device further comprises a heater operable to heat a fluid flowing through the conduit, or a cooler operable to cool a fluid flowing through the conduit.

Preferably, the sleeve comprises a gel operable to transmit ultrasound waves.

Preferably, the device is sized to be insertable into a rectum.

Preferably, the device is so shaped and configured that at least one model of ultrasound probe may be so inserted in the sleeve that when the sleeve is inserted in a body cavity ultrasound transceivers of ultrasound probe are operable to send and receive ultrasonic waves through the sleeve.

According to further features in preferred embodiments of the invention described below, the device further comprises thermal insulation, the thermal insulation being so positioned that when an ultrasonic probe is inserted in the sleeve, the thermal insulation at least partially insulates the inserted ultrasonic probe from the thermal conditioning element.

According to still further features in preferred embodiments of the invention described below the device further comprises an immobilizer operable to immobilize the device with respect to a body cavity into which the device is inserted, thereby enabling an ultrasound probe inserted in the device when the device is inserted in a body cavity to be advanced and retracted within the device without thereby causing substantial displacement of the device within the body cavity.

According to another aspect of the present invention there is provided a tissue protection device comprising a pouch having a distal end formed as pocket sized to accommodate a distal end of an ultrasound probe and an inner sheath wall and an outer sheath wall, the inner sheath wall and outer sheath wall defining a volume, and the volume comprises a semi-rigid sound-transmitting material. Preferably, the ultrasound-transmitting material is a gel.

According to further features in preferred embodiments of the invention described below, the volume further comprises a conduit for conducting a fluid through at least a portion of the volume, and the device further comprises a frame which comprises a fluid input lumen communicating with the conduit and a fluid exhaust lumen communicating with the conduit, and a pump operable to pump a fluid through the conduit. The device may further comprise a heater operable to heat the fluid and/or a cooler operable to cool the fluid. The pump may be a peristaltic pump, and the conduit may form a closed circuit.

According to further features in preferred embodiments of the invention described below, the volume further comprises an electrical heating element such as electrical resistance wires embedded in the material, or a strip of conductive rubber.

According to still further features in preferred embodiments of the invention described below, the system further comprises an immobilizer operable to immobilize the pouch with respect to a body cavity into which the pouch is inserted, thereby enabling an ultrasound probe inserted in the pouch when the pouch is inserted in a body cavity to be advanced and retracted within the pouch without thereby causing substantial displacement of the pouch within the body cavity.

Preferably, an inner surface of the inner pouch wall is so designed and constructed that an appropriately lubricated ultrasound probe inserted in the pouch slides easily forward and backward within the pouch, and an exterior surface of the outer pouch wall is so designed and constructed as to resist easy movement of the pouch with respect to walls of a body cavity when the pouch is inserted in the body cavity, the construction enabling an ultrasound probe inserted in the pouch when the pouch is inserted in a body cavity to be advanced and retracted within the pouch without causing substantial displacement of the pouch with respect to the body cavity.

According to another aspect of the present invention there is provided an ultrasound probe comprising an ultrasound transceiver operable to transmit and receive ultrasound energy along a transition path, and further comprising a thermal conditioner positioned away from the transmission path and operable to affect temperature of a surface of the probe. The thermal conditioner may be an electrical resistance heater. Preferably, the surface faces a direction towards which the probe is operable to transmit ultrasound energy. Preferably, the probe further comprises thermal insulation positioned between the thermal conditioner and heat-sensitive components of the probe, and an ultrasonic-transparent thermal diffusing layer covering the transceiver.

According to another aspect of the present invention there is provided an ultrasound probe sized for rectal insertion, comprising a thermal conditioning element operable to modify temperature of a surface of the probe while the probe is active in ultrasound imaging, an ultrasound transceiver, and a distal portion at least 2 cm long positioned distally to all ultrasound transceivers of the probe and having a surface operable to be heated by the heating element.

According to another aspect of the present invention there is provided a sheath for protecting a first tissue during ablation of a second tissue, which second tissue is distant from the first tissue, comprising a first portion comprising material substantially transparent to ultrasound waves, and a thermal conditioning element operable to influence temperature of the first tissue, and the first portion is so sized and shaped as to be operable positioned over ultrasound transceivers of an ultrasound probe when the ultrasound probe is inserted in the sheath.

The thermal conditioning element may be a heating element or a cooling element.

Preferably, the thermal conditioning element extends distally to a first position on the sheath, the sheath further comprises a distal blocking element serving to limit distal insertion of an ultrasound probe inserted into the sheath to a second position, and the second position is at least 2 cm proximal to the first position.

According to another aspect of the present invention there is provided an insertion-blocking device insertable into a temperature-influencing sheath sized to accommodate an ultrasound probe, the insertion-blocking device serving to distance a distal end of any such inserted probe from a distal end of any such sheath by at least 2 cm.

According to another aspect of the present invention there is provided a system for protection of tissue of a body conduit during ablation of tissue near the body conduit, comprising a sheath sized and shaped to fit over at least a portion of an ultrasound probe, the sheath being at least partially constructed of material transparent to ultrasound waves, a closed loop conduit operable to contain a fluid, a first portion of the conduit passing within a portion of the sheath and a second portion of the conduit being external to the sheath, and a peristaltic pump operable to be connected to the second portion of the conduit and to effect a flow in fluid contained within the conduit. Preferably, the system further comprises a heater operable to heat fluid within the conduit or a cooler operable to cool fluid within the conduit. Preferably the closed loop conduit is hermetically sealed and a fluid, preferably a liquid, is contained therein.

According to another aspect of the present invention there is provided a sleeve for warming a rectum during treatment of a prostate, the sleeve comprising a lumen defined by an inner wall, the lumen being sized to accommodate an ultrasound probe, an outer wall surrounding the inner wall, the outer and inner walls together defining a volume, a fluid hermetically contained within the volume, and a heating element contained within the volume and operable to be positioned to one side of an ultrasound probe when the ultrasound probe is inserted in the sleeve and the sleeve is inserted in a rectum.

According to another aspect of the present invention there is provided a sleeve for rectal heating sized to accommodate an ultrasound probe insertable into the sleeve, comprising a vent opening in a distal portion of the sleeve, the vent opening serving to facilitate venting of air trapped between an ultrasound probe and the sleeve when the probe is inserted into the sleeve.

According to another aspect of the present invention there is provided a method for facilitating cryoablation of prostate tissue, comprising: providing a flexible rectal wall protector insertable in a rectum, the protector being sized and shaped to accommodate an ultrasound probe inserted in a rectum alongside the protector, at least a portion of the sleeve comprises ultrasound-transmissive material operable to transmit ultrasound waves between an ultrasound probe positioned on a first side of the protector and body tissues positioned on a second side of the protector when an ultrasound probe and the protector are together positioned within a rectum, inserting an ultrasound probe and the protector into a rectum, utilizing the ultrasound probe to monitor placement of cryoprobes in a cryoablation target near the rectum, and moving the ultrasound probe longitudinally within the rectum while monitoring cryoablation activity induced by use of the cryoprobes, without substantially displacing the protector.

Preferably, the method further comprises providing a heating mechanism within the protector, and heating the protector to protect tissues of the rectum while cooling the cryoprobes.

According to another aspect of the present invention there is provided a method for facilitating cryoablation of prostate tissue, comprising providing a flexible rectal wall protector sleeve insertable in a rectum, the sleeve being sized to accommodate an ultrasound probe inserted in the sleeve, and at least a portion of the sleeve comprises ultrasound-transmissive material operable to transmit ultrasound energy between an ultrasound probe inserted in the sleeve and body tissues external to sleeve when the sleeve is inserted in the rectum and the ultrasound probe is inserted in the sleeve, inserting an ultrasound probe in the sleeve and inserting the sleeve and the ultrasound probe in the rectum, utilizing the ultrasound probe to monitor placement of cryoprobes in a cryoablation target near the rectum, and moving the ultrasound probe longitudinally within the sleeve while monitoring cryoablation activity induced by use of the cryoprobes.

Preferably, the method further comprises providing a heating mechanism within the sleeve, and heating the sleeve to protect tissues of the rectum while cooling the cryoprobes.

The present invention successfully addresses the shortcomings of the presently known configurations by providing a tissue protection device providing fluid heating or partially fluid heating, absent the disadvantages of an open fluid circulatory system.

The present invention further successfully addresses the shortcomings of the presently known configurations by providing a device for protecting the rectum from cold damage, which device enables monitoring of freezing during cooling by cryoprobes, protects the rectal wall from freezing during cryoablation of prostate tissues, and enables movement of ultrasound probe backwards and forwards within the rectum during a cryoablation procedure.

The present invention further successfully addresses the shortcomings of the presently known configurations by providing an ultrasound probe operable to heat its immediate environment and thereby to protect adjacent tissues from damage by cold, while simultaneously functioning as an instrument of ultrasonic visualization.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a and 1b are simplified schematics of side and cross sectional views respectively of a rectal warming sleeve, according to an embodiment of the present invention;

FIG. 1c is a simplified schematic presenting a detail of the configuration presented by FIGS. 1a and 1b, according to an embodiment of the present invention;

FIG. 2 is a simplified schematic of a pumping and heating apparatus for use with a rectal warming apparatus, according to an embodiment of the present invention;

FIG. 3a is a simplified schematic of a rectal warmer operable to circulate a heated warming fluid in a closed and sealed flow circuit; according to an embodiment of the present invention;

FIGS. 3b and 3c present cross sectional and side views, respectively, of an optional alternative construction of the sealed rectal warmer presented by FIG. 3a, according to an embodiment of the present invention;

FIG. 3d is a simplified schematic of an alternative construction of a sealed rectal warmer similar to that presented in FIGS. 3a-3c, designed for use with, but unattached to, an ultrasound probe, according to an embodiment of the present invention;

FIG. 4 is a simplified schematic of a chemical-reaction rectal warmer sleeve according to an embodiment of the present invention;

FIG. 5 is a simplified schematic of a concentric flow heater or cooler sleeve, according to an embodiment of the present invention;

FIGS. 6a and 6b are simplified schematics of a convection-flow rectal warmer sleeve, according to an embodiment of the invention;

FIG. 7 is a simplified schematic of a rectal warming sleeve which comprises an internal heater, according to an embodiment of the present invention;

FIGS. 8a and 8b are simplified schematics of a rectal warmer sleeve utilizing a conductive rubber electrical resistance heating element, according to an embodiment of the present invention;

FIGS. 9a and 9b are simplified schematics of a conductive liquid rectal warmer, according to an embodiment of the present invention;

FIG. 10 is a simplified schematic of a rectal protection sleeve designed to enable free movement of an ultrasound probe within a rectum without compromising ultrasound viewing of a prostate and without compromising protective heating of rectum tissues;

FIG. 11 is a simplified schematic of a rectal protection device which comprises a semi-rigid frame having branches joined by a flexible pouch, according to an embodiment of the present invention;

FIG. 12 is a simplified schematic of an additional view of the rectal protection device of FIG. 11, according to an embodiment of the present invention;

FIG. 13 is a simplified schematic of a cross-sectional view of the device of FIG. 11, according to an embodiment of the present invention;

FIG. 14 is a simplified schematic of an alternate construction of the device of FIG. 11, utilizing electrical heating, according to an embodiment of the present invention;

FIG. 15 is a simplified schematic of an ultrasound probe, showing typical positioning of transceivers, according to methods of prior art;

FIG. 16 is a simplified schematic of an ultrasound probe comprising a heating element, according to an embodiment of the present invention;

FIG. 17 is a simplified schematic showing a cross-sectional view of the ultrasound probe of FIG. 16, according to an embodiment of the present invention;

FIG. 18 is a simplified schematic of an ultrasound probe with heater or cooler, having an extended distal portion, shown in a first position, according to an embodiment of the present invention; and

FIG. 19 is a simplified schematic of the probe of FIG. 18, shown in a second position, according to an embodiment of the present invention.

### DESCRIPTION OF THE PREFERRED EMBODIMENTS

The present invention is of devices and methods for protecting the wall of a body cavity during thermal ablation of tissues near that body cavity. In particular, the present invention relates to devices for heating or cooling a rectal wall during cryoablation of prostate tissues while enabling use of a rectal ultrasound probe to visualize the prostate area, the devices enabling free movement of the ultrasonic probe within the rectum during imaging, with continuous protection to rectal tissues.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The present invention is principally described in the following with reference to an exemplary context, namely that of protective heating of a rectal wall during cryosurgical treatment prostate tissue, and to ultrasound visualization of the target tissues by means of an ultrasound probe inserted in the rectum. It is to be understood that invention is not limited to that exemplary context. The invention is, in general, relevant to thermal protection of any tissues undergoing thermal treatment in proximity to an ultrasound probe used to visualize tissues. Thus, methods and devices of the present invention are relevant to treatment of the vagina, the esophagus, to treatment of additional locii adjacent to the rectal cavity, to treatment of external walls of a uterus etc. For example, one might use the devices presented here to warm the vagina and/or the rectum while treating posterior fibroids of the uterus, or cervical cancer.

Similarly, although the following discussion is primarily addressed to the exemplary context of protecting tissues from damage by cold, all of those embodiments below which refer to protecting tissues using an object heated by a flow of heated fluid during cryosurgery are also useful in protecting tissues by cooling utilizing a flow of cooled fluid during heat-producing forms of ablative surgery. Although for simplicity of exposition most of the discussion hereinbelow is couched in terms of protecting tissue by heating in the exemplary context of rectal ultrasound and prostate cryosurgery, it is to be understood that the invention is equally relevant to protecting tissue by cooling, and the various references to devices and methods for protecting tissues by means of pumped flow of heated fluid are to be read as teaching as well the possibility of protecting those tissues by pumped flow of cooled fluid, and to imply obvious modifications to the configurations described (e.g. substitution of a cooling element in place of a heating element where appropriate). For example, it is noted that substituting cooling devices in place of heating devices in descriptions of embodiments presented hereinbelow which specify tissue protection by use of heated fluids results in embodiments useable to protect e.g. the rectal wall against high temperatures produced by heat ablation of the prostate, by cooling the rectal wall during such thermal treatment. Thus, all the embodiments presented below having reference to fluid-flow heating are to be understood as also teaching the possibility of fluid-flow cooling to protect tissues, whether or not such a configuration and such a use is explicitly mentioned.

References herein and in the claims below to “thermal conditioning element(s)” and “temperature-controlling elements” are to be understood to refer to heating elements and also to cooling elements. A “thermal conditioner” is a device which comprises a thermal conditioning element.

It is expected that during the life of this patent many relevant cryoprobes and other thermal treatment probes will be developed, and the scope of the term “cryoprobe” is intended to include all such new technologies a priori. Similarly, it is expected that during the life of this patent many relevant ultrasound probes and other forms of visualization probes capable of insertion in body cavities will be developed, and the terms “ultrasound probe” and “ultrasonic probe” are intended to include all such new technologies a priori.

In discussion of the various figures described hereinbelow, like numbers refer to like parts.

Cryoablation of the prostate is preferably monitored using a rectal ultrasonic probe. Consequently, in the drawings and in the following discussion thereof, embodiments of the invention are presented together with a rectal ultrasonic probe. However, it should be noted that the ultrasound probe is not essential to the protective function of the devices presented, and may be replaced with a structural member for insertion of a warmer into the rectal cavity, or may be absent entirely. In particular, it is a specific advantage of various embodiments presented hereinbelow that an ultrasound probe or other member used during introduction of a heating element into a rectum may subsequently moved within that rectum or removed from that rectum without adversely affecting the protective functionality provided by the device.

When a rectal ultrasonic probe is used, it is preferable to avoid interruption of the view of that probe by structures that absorb, reflect, or otherwise interfere with the transmission of ultrasound waves directed towards or reflected from the treated organ. Elements of the design of the device here presented provide for undisturbed ultrasonic imaging during rectal warming. Of course, structural features and limitations designed to provide undisturbed ultrasonic imaging are unnecessary if the pictured rectal ultrasonic probe is not present or not in use.

When using the warming device with a rectal ultrasonic probe, it is preferable that a suitable gel be used for lubrication and to facilitate ultrasonic wave transmission. The gel is preferably applied to any surfaces through which ultrasound waves are to be transmitted. In particular, it is advisable to provide ultrasonic-facilitation gel to that surface of the warming device which is in contiguous to the rectal wall and facing the prostate.

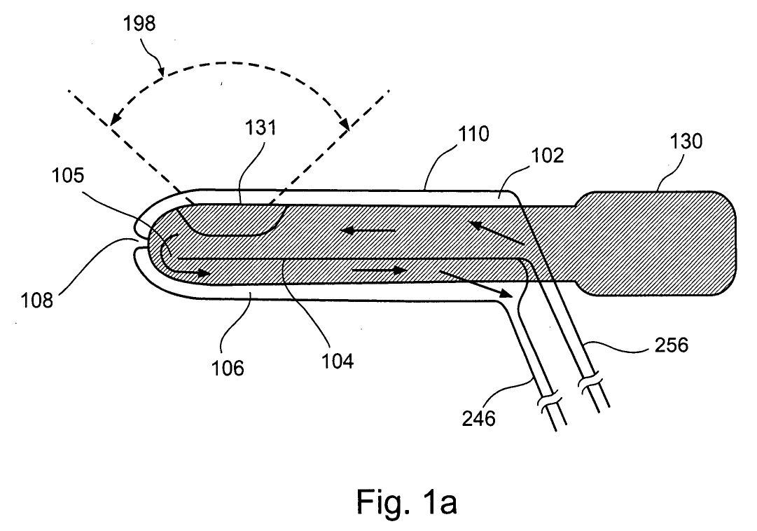

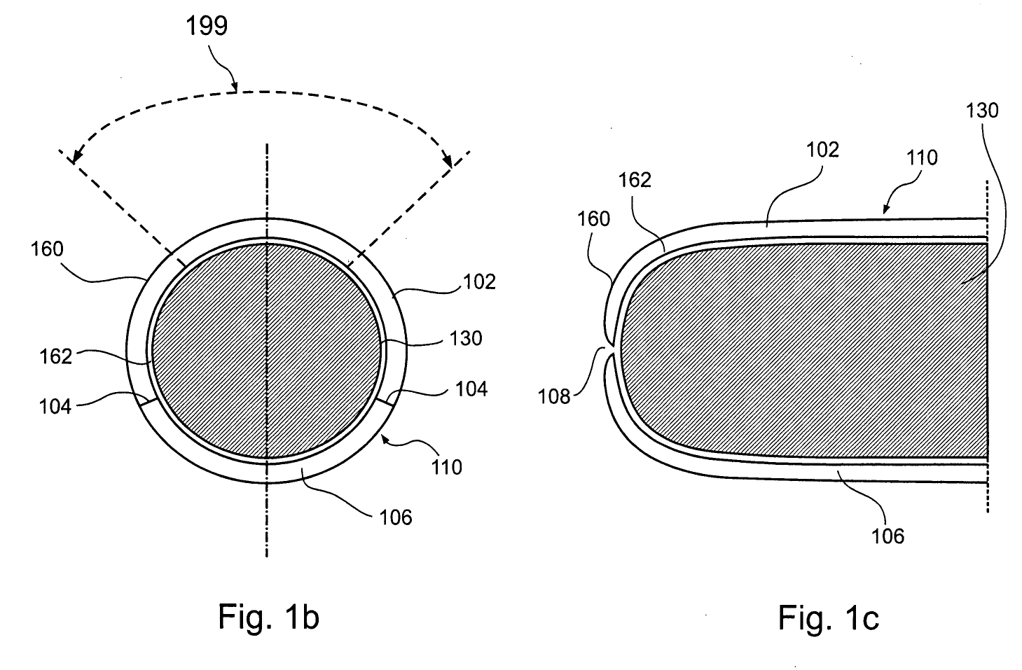

Attention is now drawn to FIGS. 1a, 1b, and 1c, which are simplified schematics of a rectal warming sleeve according to an embodiment of the present invention.

FIG. 1a presents a side view of a rectal warming sleeve 110, designed for warming and protecting the rectal wall during cryoablation of the prostate.

Rectal ultrasonic probe 130 comprising ultrasonic transceiver 131 is used for monitoring cryoablation (or other thermal treatment) of the prostate (or other organ). Probe 130 is may be any commercially available rectal ultrasound probe. In a recommended method of use, ultrasound gel is applied to probe 130, after which probe 130 is inserted into warming sleeve 110, gel is applied to the exterior of warming sleeve 110, and the combined probe/sleeve assembly is inserted into a rectal cavity.

In FIGS. 1a and 1b, the field of view (FOV) of a rectal ultrasonic probe 130 is schematically marked by heavy dashed lines and the viewing angles are marked 198 and 199 for the axial and transverse directions respectively. Sleeve 110 is preferably made of flexible, ultrasound-transparent material such as plastic or rubber. Alternatively, only those portions of sleeve 100 which are positioned in front of ultrasonic transceiver 131 may be made of ultrasound-transparent material. Thus, for uninterrupted ultrasonic imaging, homogeneous ultrasonic-transparent materials are used for all device parts positioned within the FOV.

Sleeve 110 may be a disposable unit, or it may be reused. If reused, a protective covering such as a condom may be used over the assembly.

Sleeve 110 comprises a warming-fluid compartment 102 partially separated by a septum 104 from fluid return path 106. The two compartments are connected by at least one discontinuity 105 in the septum, preferably near or at the distal end of sleeve 110. The assembly is preferably inserted into the rectum in such a position that warming compartment 102 is positioned over transceiver 131 and facing the prostate.

An optional venting orifice 108 is provided for venting the internal lumen sleeve 100 during insertion of probe 130 into sleeve 110, thus easing insertion of probe 130 into sleeve 110 and reducing the risk of trapping air within sleeve 110, since trapped air within sleeve 110 might subsequently impede ultrasonic monitoring.

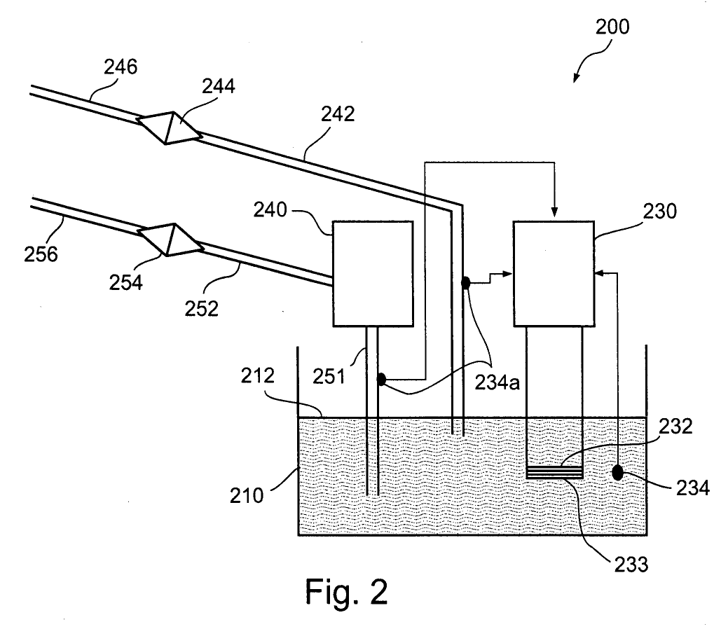

Flexible liquid pipe 256 is connected to warming compartment 102, and flexible liquid pipe 246 is connected to return path 106. Pipe 256 is used for providing a warming liquid, such as water, saline or oil to sleeve 100, and pipe 246 is used to exhaust the used warming fluid therefrom. Warming fluid is provided from, and exhausted into, an external fluid circulator 200 depicted in FIG. 2. Fluid flow is preferably as shown by the arrows within compartment 102 and path 106, yet alternatively, flow direction may be reversed.

FIG. 1b presents a cross-sectional view of the configuration presented in FIG. 1a, taken along the dot-dash vertical line seen near the center of FIG. 1a.

Warming sleeve 110 is here seen to comprise an outer wall 160, and inner wall 162. Warming compartment 102 is seen to be divided from fluid return path 106 by septum 104.

For clarity, an optional gel layer, preferably provided between rectal ultrasonic probe 130 and inner layer 162, is not shown in the Figure.

FIG. 1c presents a detail view of a distal portion of sleeve 110, showing optional venting orifice 108 and various other elements of sleeve 110 with increased clarity. The Dotted line in FIG. 1b indicates the location of the cross-section depicted in FIG. 1c.

Attention is now drawn to FIG. 2, which is a simplified schematic of a pumping and heating apparatus, according to an embodiment of the present invention. Pumping and heating apparatus 200 presented in FIG. 2 is usable with rectal warming sleeve 110.

Pumping and heating apparatus 200 is connectable to warming sleeve 110 via flexible pipes 246 and 256. Pump 240 pumps warm liquid to rectal warmer 110, while heater 232 maintains a desired temperature in the pumped fluid.

Preferably, the pumped fluid is a liquid. In the exemplary embodiment shown. in FIG. 2, pumping and heating apparatus 200 comprises an open liquid container 210 which acts as a reservoir for circulating liquid.

Preferably, heater 232 is thermostatically controlled to maintain the circulating liquid within safe and desirable pre-set temperature limits. Heating element 232 may be an electric resistive heater or other heater, such as a heat pump or a thermoelectric couple. Heater controller 230 regulates electric current through heating elements of heater 232 in response to temperature readings from thermal sensor 234 within the liquid container 210. Alternatively or additionally, one or more optional thermal sensors 234a may be installed to provide temperature reading at liquid input and output of the apparatus.

Alternatively, in place of heating element 232 another temperature-controlling element may be provided, for example a cooling element 233 such as a thermoelectric cooler (TEC). In this case, controller 230 may control cooling of the circulating liquid.

Pump 240 draws liquid from liquid container 210 through inlet 251 and pumps it to the warming sleeve through outlet pipe 252. The liquid returns to liquid container 210 through liquid return pipe 242.

Preferably, outlet pipe 252 is connected to flexible liquid pipe 256 through a quick-connect connector 254, and liquid return pipe 242 is connected to flexible liquid pipe 246 through quick-connect connector 244.

Optional safety features may be installed in heating apparatus 200 or sleeve 100, to monitor and, when appropriate, alert or correct conditions such as liquid overpressure, impeded liquid flow, and overheating.

A system comprising sleeve 110 and heating/pumping apparatus 200 can protect the rectal wall during cryoablation of prostate tissues, while enabling continuous ultrasound monitoring of the cryoablation process. Similarly, a system incorporating cooling element 233 can protect the rectal wall during heat-producing ablation of the prostate. The system as described is inexpensive to build and to use, as re-useable components such as apparatus 200 and ultrasound probe 130 may easily be combined with a sleeve 110 which may be presented in disposable format for one-time use.

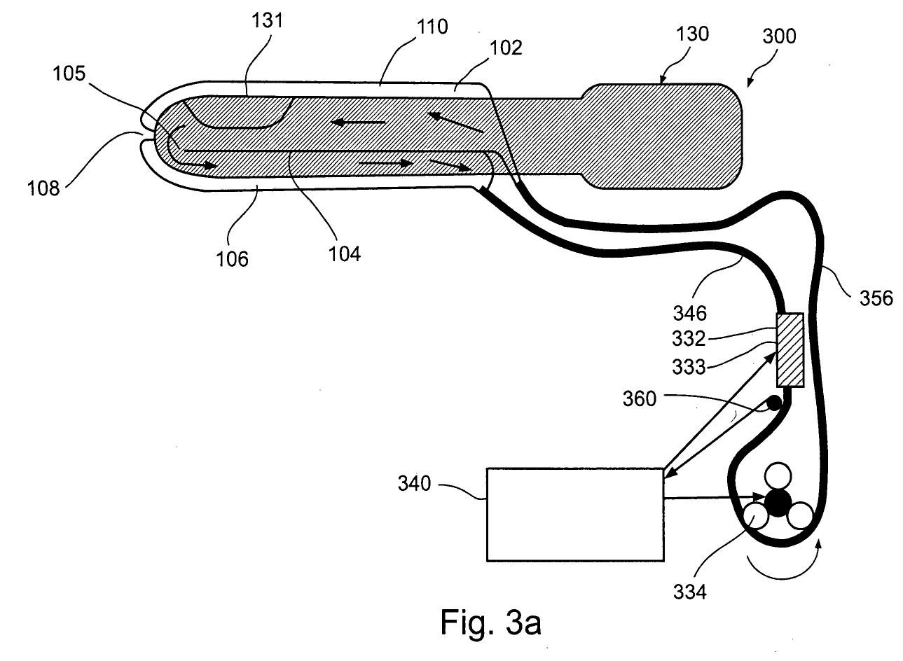

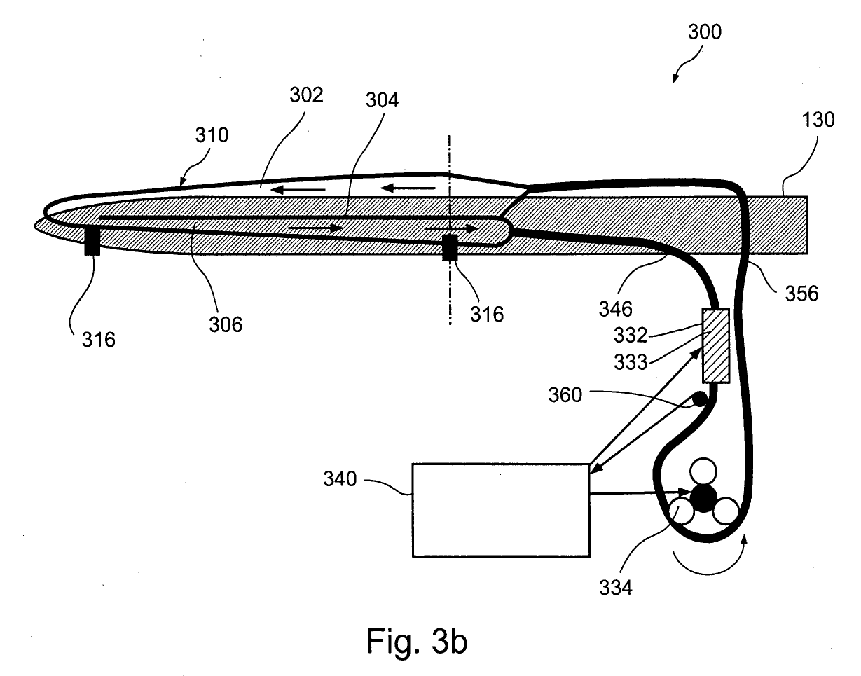

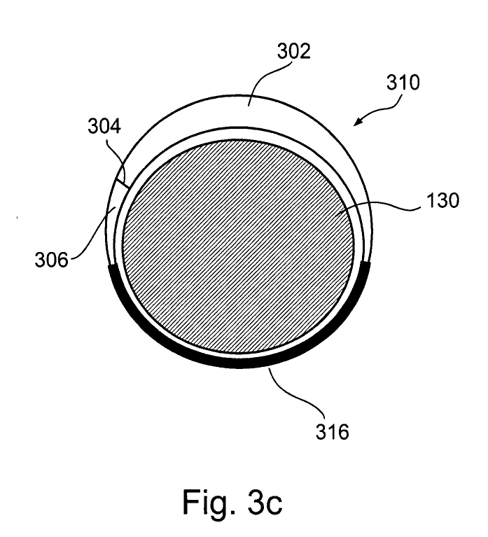

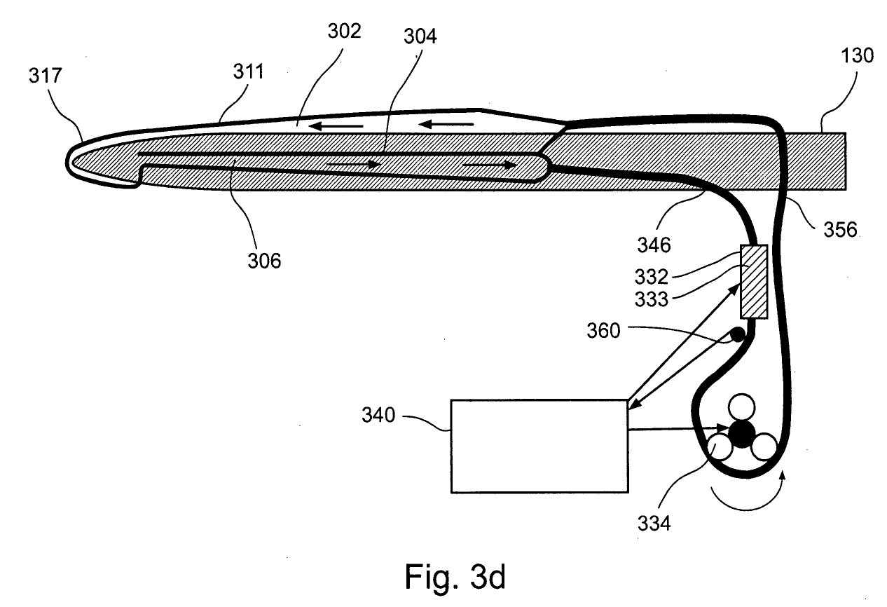

Attention is now drawn to FIG. 3a, which is a simplified schematic of a rectal warmer operable to circulate a heated warming fluid in a closed and sealed flow circuit, according to an embodiment of the present invention.

FIG. 3a presents a side view of rectal warmer wormer 300. Rectal warmer 300 comprises a rectal warming sleeve 310, which in turn comprises a warming compartment 302 separated from a fluid return compartment 306 by a septum 304.

Flexible pipes 356 and 346 connect to warming compartment 302 and fluid return compartment 306 respectively, and are used to circulate a warm fluid (in a preferred embodiment, a warm liquid) through sleeve 310. Liquid circulation is maintained by pump 334 while temperature of the circulating liquid is maintained by heating element 332. Optional controller 340 controls liquid flow and temperature, receiving temperature reports from optional temperature sensor 360, and sending commands to pump 334 and heater 332.

In an alternative embodiment of the invention, a cooling element 333, such as a thermoelectric cooler, may be used in place of heater 332, and be controlled by controller 340 to effect controlled cooling of tissues endangered by heat-producing (e.g. radio-frequency) ablation. Further alternatively, a combined heating/cooling element might be used, in this embodiment and in other embodiments described herein, and a controller such as controller 340 might be operable to selectively switch between heating and cooling of the circulating fluid.

Preferably, pump 334 is a peristaltic pump. In this embodiment, pipes 346 and 356 are in a form of one contiguous flexible hose, which is threaded through peristaltic pump 334. Thus, FIG. 3a presents a closed-loop fluid flow system which presents undoubted advantages of simplicity of use, including easy assurance of the sterility of fluids thus introduced into the body.

A similar but slightly altered embodiment of the present invention is presented in FIGS. 3b and 3c. Since an intended use for the present embodiment is to protect the rectal wall during thermal ablation of the prostate, it is possible to warm (during cryoablation) or cool (e.g. during High Intensity Focused Ultrasound ablation) only those parts of the rectal wall which are susceptible to thermal damage, these being the parts of the rectal wall positioned between the ultrasound probe (or other core structure inserted in the rectum) and the prostate. FIGS. 3b and 3c present an embodiment of the present invention similar to that presented in FIG. 3a, but such that portions of the rectal wall near the cryogenically treated organ are warmed by the rectal warmer, while parts of the rectal wall away from the thermally treated organ are not warmed by it. FIGS. 3b and 3c show a rectal warmer similar to that presented in 3a, yet designed to circulate a warming fluid only over one side or portion of the ultrasound probe, yet not over all sides. As may be seen in FIGS. 3a and 3b, straps 316 may be provided to attach rectal warming sleeve 310 to rectal ultrasonic probe 130 in such manner that the portion of probe 130 facing the prostate is warmed, and the side of the probe not facing the prostate is not warmed and indeed may not be covered by sleeve 310.

In a preferred embodiment, heating element 332, peristaltic pump 334, controller 340 and optionally thermal sensor 360 are embodied as a reusable unit, while sleeve 310 with a hose (346 combined with 356) and optional straps 316 is presented as a sterile disposable unit for one-time use, preferably sold pre-filled with liquid. To assemble the rectal warmer, sleeve 310 is attached to rectal ultrasonic probe 130, and the hose (combining 346 and 356) is threaded through heating element 332 and peristaltic pump 334 and caused to make thermal contact with thermal sensor 360.

The liquid supplied in this closed-loop system is preferably degassed to prevent circulation of bubbles, since bubbles in the circulating liquid might interfere with the ultrasonic imaging process. The supplied liquid (and liquids used in other embodiments of the present invention) should be selected to be safe to use within the body. Preferred liquids include saline, water, and appropriate oils.

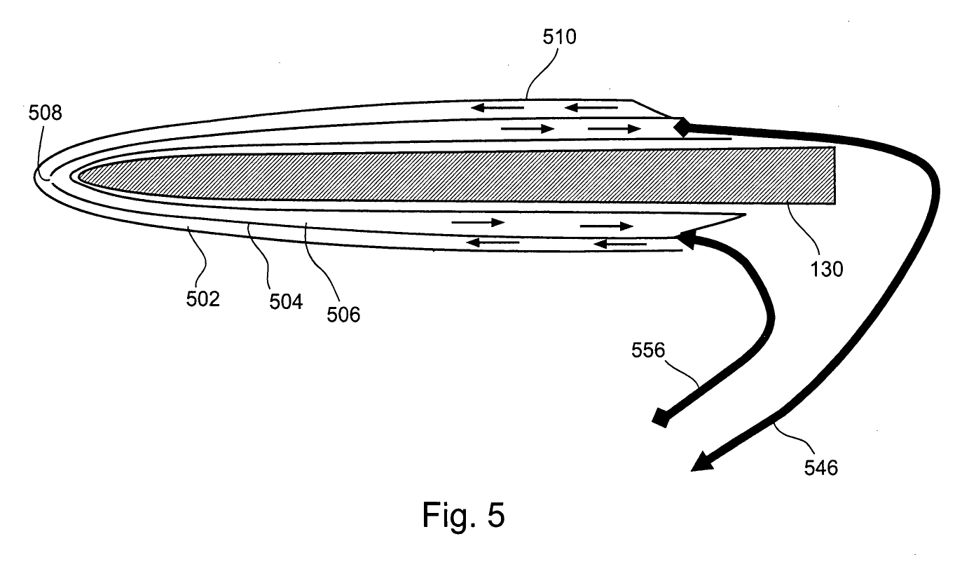

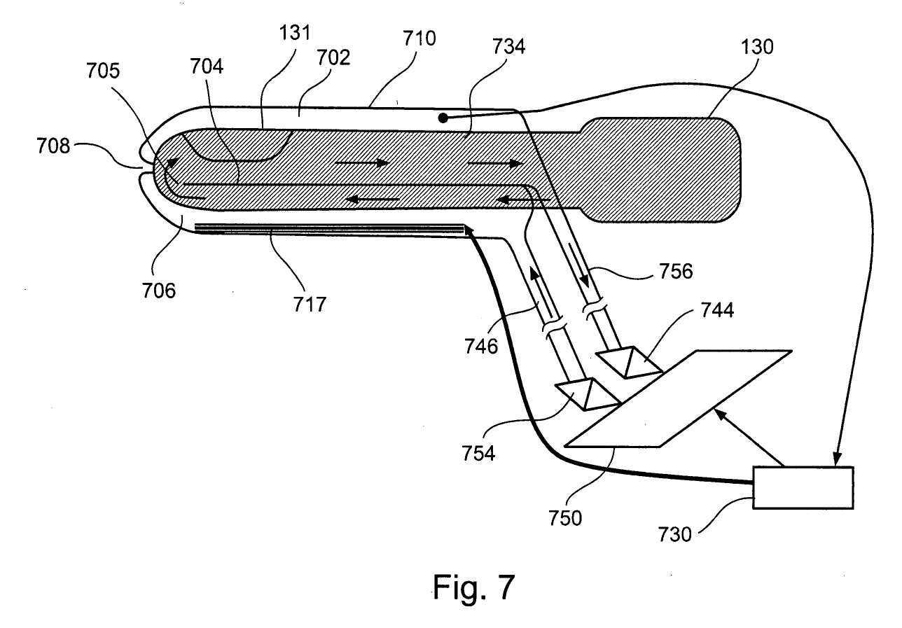

It should be noted that the specific form of sleeve 310 presented in FIGS. 3a, 3b, and 3c is exemplary only. Other forms incorporating the principles presented herein may be used for rectal warmer sleeves, sealed (as in FIG. 3) or unsealed (as in FIGS. 1 and 2). Additional exemplary shapes are presented in FIGS. 5 and 7 and elsewhere hereinbelow.

In FIGS. 3a and 3b, small arrows are provided to show preferred (optional) liquid flow direction. The dashed line in FIG. 3b identifies the position of the cross section seen in FIG. 3c.

In addition to the advantages noted above with respect to the embodiments presented in FIGS. 1 and 2, the embodiments presented in FIGS. 3a, 3b, and 3c present additional advantages of simplicity of use. The system provided is spill-proof and can easily be made leak-proof. The system is relatively inexpensive to use, in that the more complex mechanical and/or electronic parts are presented in re-useable format whereas those portions inserted into the rectum can be designed and constructed for one-time use. The sealed presentation of these embodiments simplifies use and enables to supply a liquid free of gas bubbles and consequently highly compatible with ultrasound imaging processes.

Attention is now directed to FIG. 3d, which is a simplified schematic of a rectal warmer designed for unattached coordinated use with an ultrasonic probe, according to an embodiment of the present invention. Sleeve 311 presented in FIG. 3d is similar in all respects to sleeve 310 presented in FIG. 3b, with the exception that straps 316 are absent, and the distal end of sleeve 311 is formed as a shallow pouch 317 sized to fit over the distal end of probe 130. Sleeve 311 is designed to be used together with a probe 130 yet without being physically attached thereto. According to a recommended method of use, for purposes of insertion pouch-like formation 317 at a distal end of sleeve 311 is fitted over a distal end of probe 130. Probe 130 is then inserted deeply into a rectum, thereby installing sleeve 311 in that rectum in fully inserted position. Probe 130, being unattached, is then free to move forward and backward within that rectum at the convenience of the surgeon, while sleeve 311 remains in place and is enabled to function in protecting rectal tissues from the effects of nearby ablation procedures, regardless of the position of rectal probe 130. In yet a further alternative construction, straps 316 may be provided with sleeve 311, yet be designed for a very loose contact with probe 130, such that they facilitate the insertion process but do not afterwards impeded freedom of movement of probe 130 with respect to inserted sleeve 311.

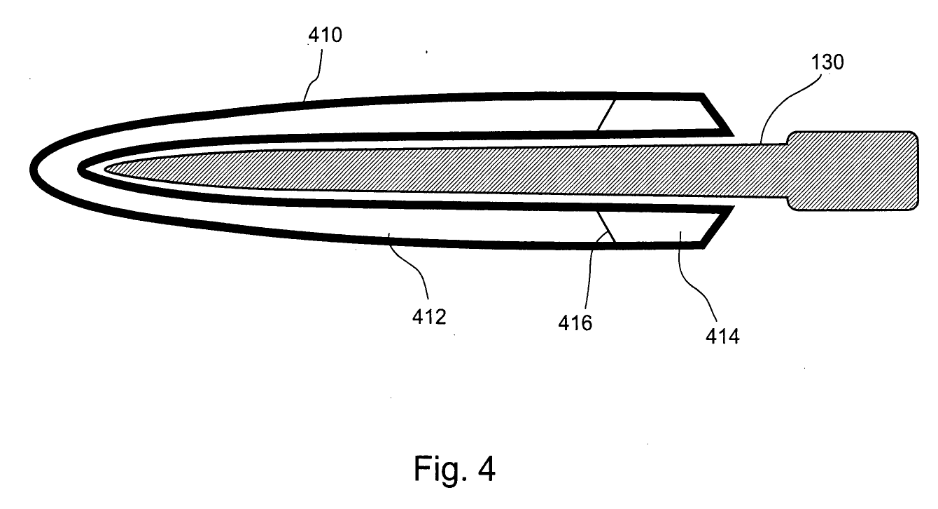

Attention is now drawn to FIG. 4, which is a simplified schematic of a chemical-reaction rectal warmer sleeve for protecting a rectal wall during cryoablation of nearby organs, according to an embodiment of the present invention. Chemical-reaction sleeve 410 uses chemical reaction between substances contained within sleeve 410 to heat the rectum, thereby protecting it from damage by freezing.

Chemical reaction rectal warmer sleeve 410 comprises a first compartment 412 containing a first chemical compound and a second compartment 414 containing a second chemical compound, compartments 412 and 414 being separated by a breakable septum 416.

During storage and prior to use, breakable septum 416 prevents mixing of the first and second chemical compounds. When it is desired to utilize chemical reaction rectal warmer sleeve 410, the user compromises the integrity of breakable septum 416 by applying pressure on first or second compartments, which pressure breaks septum 416 and allows mixing of first and second chemical compounds. Mixing first and second chemical compounds initiates an exothermic chemical reaction that produces heat, warming sleeve 410. In some cases it may be advantageous to present a plurality of first compartments 412 interspersed with a plurality of second compartments 414 separated by a plurality of septa 416, so as to facilitate mixing of the first and second chemical compounds when septa 416 are broken. Alternatively or additionally, portions of the first chemical compound, or of first and second chemical compounds, can be presented in timed-release encapsulation, which encapsulation breaks down at a pre-set rate, thereby enabling to control production of heat and extend the heating process over a predictable lapse of time.

The embodiments presented in FIG. 4 presents advantages of simplicity of use. No electrical connections are required, no spillable liquids are involved, the device has no elements external to warming sleeve 410 itself, and has no re-useable parts.

Attention is now drawn to FIG. 5, which is a simplified schematic of a rectal warming sleeve having a concentric fluid flow path, according to an embodiment of the present invention.

FIG. 5 presents a sleeve 510 which is similar to sleeves presented in FIGS. 1 and 3, yet having an alternative form of flow path for the warming fluid circulated therein. As may be seen in FIG. 5, sleeve 510 is a concentric flow heater (or cooler) sleeve and comprises of an outer warming (or cooling) compartment 502 and an inner fluid return path 506. Warming/cooling compartment 502 and fluid return path 506 are separated by septum 504. When circulating, fluid flows from outer compartment 502 to inner return path 506 by passing through connecting orifice 508, which is preferably located (as shown) at or near the distal end of sleeve 510.

Flexible liquid input pipe 556 is provided as an input conduit for conducting a warm circulating fluid, preferably a liquid, into warming compartment 502. Liquid exhaust pipe 546 is provided for exhausting circulated liquid from fluid return path 506. Circulating warm fluid through the fluid flow path shown by the arrows in FIG. 5 provides thermal protection for the rectal wall during cryogenic treatment of organs near the rectum, for example during cryogenic treatment of the prostate. Circulating liquid may be warmed and pumped by apparatus similar to that presented in FIGS. 2 and 3a, and discussed hereinabove. Similarly, thermal protection of the rectum wall during hot thermal treatment of organs near the rectum (e.g. during High Intensity Focused Ultrasound treatment) may be provided by pumping cooled water through sleeve 510. The small arrows in FIG. 5 present a preferred direction for fluid flow. The embodiment of FIG. 5 is advantageous in that it provides relatively more uniform heat than the embodiments of FIGS. 1 and 3, previously described.

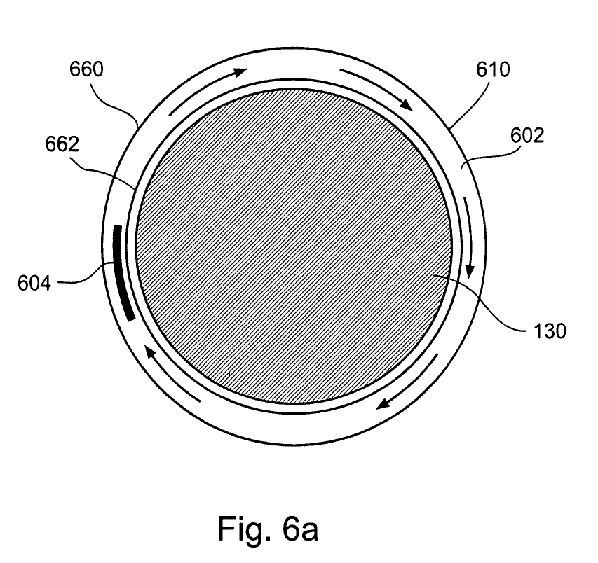

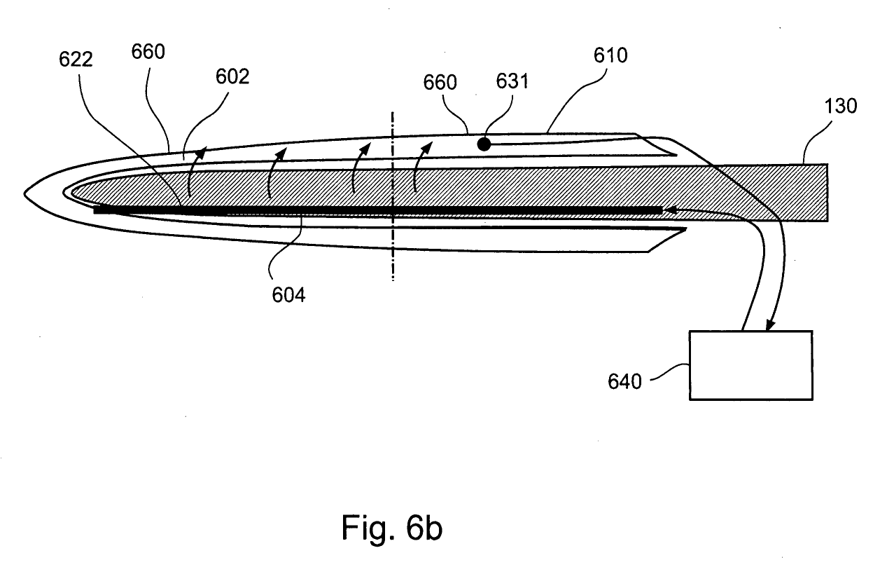

Attention is now drawn to FIGS. 6a and 6b, which are simplified schematics presenting views of a convection-flow rectal warmer sleeve, according to an embodiment of the present invention.

FIG. 6a presents a cross sectional view and FIG. 6b a side view of a rectal warmer sleeve 610 which utilized convection to distribute heat. The dashed line in FIG. 6b shows the position of the cross section presented in FIG. 6a.

Convection flow rectal warmer sleeve 610 is useful for protecting the rectal wall during thermal ablation of neighboring tissues, and is advantageous by virtue of its simplicity. Convection flow rectal warmer sleeve 610 provides for circulation of a warming fluid within a sleeve surrounding or partially surrounding an ultrasound probe inserted in the rectum, without requiring external pumps or attachment of liquid sources. Indeed, sleeve 610 has no moving parts.

Convection flow rectal warmer sleeve 610 is shown in these figures with rectal ultrasonic probe 130 inserted therein. Sleeve 610 comprises a liquid compartment 602 defined by outer layer 660 and inner layer 622. Liquid compartment 602 is preferably sealed, and pre-filled with a liquid such as water, saline or oil. The liquid filling compartment 602 is preferably degassed, so as to exclude gas bubbles that might otherwise interfere with ultrasonic imaging.

Heating element 604 is located within or in thermal contact with liquid compartment 602, preferably in non-symmetric position such as that shown in FIGS. 6a and 6b. When heating element 604 is operated to heat liquid contained in compartment 602, convection current is created within compartment 602. Heating element 604 may be an electric resistance heater or other heater. Power is supplied to heating element 604 preferably under control of an optional heater controller 640. Controller 640 receives temperature information from one or more an optional thermal sensors 631, which may be used to sense temperature of the rectal wall and/or temperature of circulating liquid within compartment 602. Sensors 631 thus enable controller 640 to maintain temperatures of liquid within compartment 602 and/or temperatures of the rectal wall within a pre-set range.

Convection currents, caused by density differences brought about when liquid in sleeve 610 is heated by heater 604 and then progressively cooled by indirect contact with cooled rectal tissues, serve to distribute heat produced by heater 604 throughout sleeve 610. Arrows are used in FIGS. 6a and 6b to show direction of flow of heat-induced convection current. Asymmetric placement of heater 604 serves to enhance production of convective liquid flow. Positioning of heater 604 to one side of ultrasonic probe 130, rather than above probe 130, assures that heating element 604 will be outside the field of view of the ultrasonic imager, thereby enabling unimpeded ultrasonic imaging. (Ultrasonic transceivers are not shown in FIG. 6, but are assumed to be embodied within probe 130 and to be pointing upwards towards the prostate, which is typically positioned above the rectum during prostate surgery.), Sleeve 610 preferably comprises safety features such as detectors of excessive current in heating element 604, high or low temperature shutdown or alert, current leakage detection, liquid leakage detection, and so on, all of which serve to reduce patient risk and to increase reliability.

Sleeve 610 is preferably supplied as a sterile disposable unit equipped with connecting cable which interfaces with a re-useable heater controller 640. Alternatively, sleeve 610 may be presented in reusable format. Optionally, gel is applied to surfaces of sleeve 610 for ease of insertion and to facilitate ultrasonic wave transmission. Optionally sleeve 610 is enveloped in a condom before insertion into the rectal cavity.

Attention is now directed to FIG. 7, which is a simplified schematic of a rectal warming sleeve which comprises an internal heater, according to an embodiment of the present invention.

FIG. 7 provides a side view of a rectal warming sleeve 710 which comprises an internal heater 717 and provision for an externally provided pumped (and optionally heated) circulating liquid. Warming sleeve 710 is useful for warming and protecting the rectal wall during cryoablation of the prostate. Small arrows in FIG. 7 present a preferred direction for liquid flow.

Sleeve 710 comprises a rectal wall warming compartment 702 partially separated by septum 704 from heater-containing liquid-heating compartment 706. The two compartments are connected by at least one discontinuity 705 in the septum, preferably near or at the distal end of the sleeve. The assembly is preferably inserted in a rectum in such position that warming compartment 702 is positioned facing the prostate. Internal heating element 717 inside (or in thermal contact with) heater compartment 706 is used for warming the liquid contained therein, which liquid preferably circulates therefrom into warming compartment 702, thereby aiding to maintain liquid in warming compartment 702 at a desired temperature. Internal heating element 717 may be a electric resistive heater or other heater.

Flexible liquid pipes 756 and 746 connected to warming compartment 702 and heater compartment 706 are used for circulating warming liquid such as water, saline or oil, pumped by an external circulator 750. A preferred liquid flow direction, shown by arrows, is from liquid heating compartment 706 to rectal wall warming compartment 702, but that direction may be reversed.

Optional hose connections 744 and 754 connect the flexible liquid pipes 746 and flexible liquid pipe 756 to an external circulator 750. External circulator 750 may comprise a centrifugal pump, peristaltic pump, piston pump, or other pump.

Rectal ultrasonic probe 130 may be inserted into sleeve 710 and used for monitoring thermal treatment of the prostate. Preferably, a commercially available probe is used. Preferably, gel is applied to probe 170 as it is inserted into warming sleeve 710. Preferably, gel is applied to warming sleeve 710 prior to insertion into the rectal cavity.

Sleeve 710 may be a disposable unit, or it may be reused. If reused, a protective covering such as a condom may be used over the assembly.

Sleeve 710 is preferably made of flexible, ultrasound transparent material such as plastic or rubber. Alternatively, only parts of the sleeve in front of the ultrasound transceiver within probe 130 are made of ultrasound transparent material.

Optional venting orifice 708 (similar to orifice 108 shown in detail in FIG. 1c) may be used for venting the internal lumen sleeve 710 during insertion of probe 130 into sleeve 710, thus easing insertion and reducing the risk of trapping air within sleeve 710, which might impede ultrasonic imaging.

A controller 730 receives temperature readings from thermal sensor 734 and regulates heat generated by the heating element 717 and/or pumping action of circulator 750. Preferably, thermal sensor 734 is located in or near rectal wall warming compartment 702.

Optionally, additional thermal sensors monitor temperature in other locations.

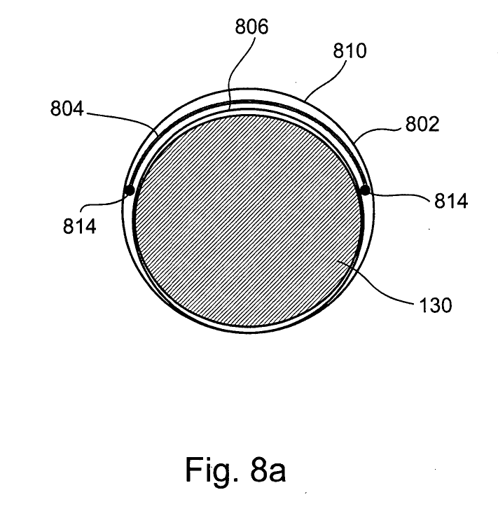

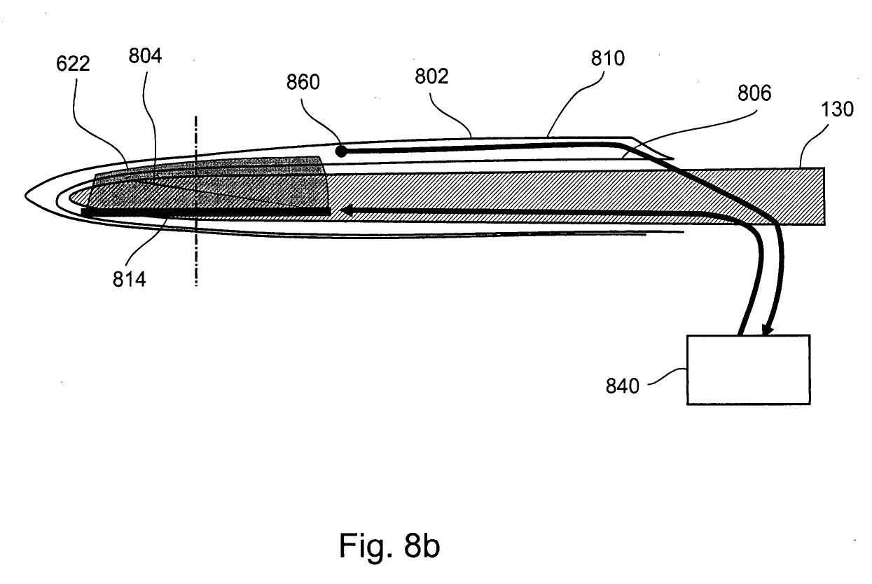

Attention is now drawn to FIGS. 8a and 8b, which are simplified schematics of a rectal warmer sleeve utilizing a conductive rubber electrical resistance heating element, according to an embodiment of the present invention.

FIG. 8a presents a cross sectional view and FIG. 8b a side view of a rectal warmer sleeve 810 useful for protecting a rectal wall during thermal ablation of nearby organs. A dashed line in FIG. 8b indicates the longitudinal position of the cross section shown in detail in FIG. 8a.

Conductive rubber rectal warmer sleeve 810 is shown in FIGS. 8a and 8b together with a rectal ultrasonic probe 130 inserted therein. Sleeve 810 comprises a flexible electrical resistive heating element 804 made of electrically resistive ultrasonic transmitting material such as Velostat™ Electrically Conductive Film, as supplied by the 3M company and described at Internet site http://www.3m.com/. Electric current is applied to the flexible electrical resistive heating element 804 by applying voltage to electric connector wires 814, causing the heating element 804 to produce thermal energy.

Preferably, an outer electrical insulator layer 802 and optionally inner electrical insulator layer 806 isolate heating element 804, reducing risk of electrocution. Layer 806 may also optionally provide thermal insulation to protect probe 130 from damage by heat.

Heating element 804 is receives power regulated by thermal controller 840. An optional thermal sensor 860 connected to heater controller 840 is used for sensing and reporting rectal wall temperature, enabling controller 840 to regulate that temperature to within a pre-set range.

Safety features such as detectors of excessive current in heating element 804, high or low temperature shutdown or alert, a current leakage detector, etc. may be implemented to reduce patient risk and to increase reliability.

Since the embodiment presented in FIGS. 8a and 8b utilizes no liquids and has no moving parts, it presents advantages of low cost and simplicity of construction.

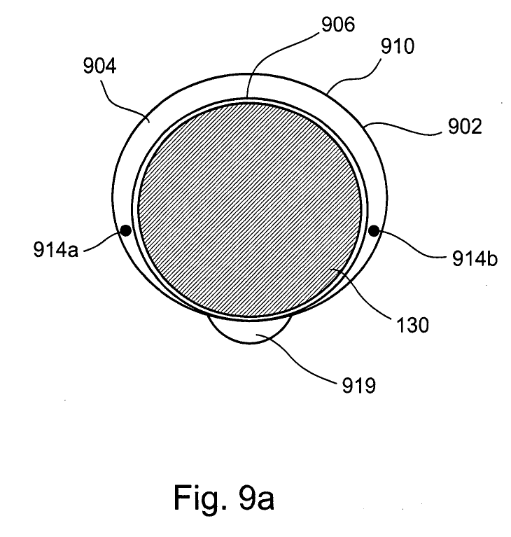

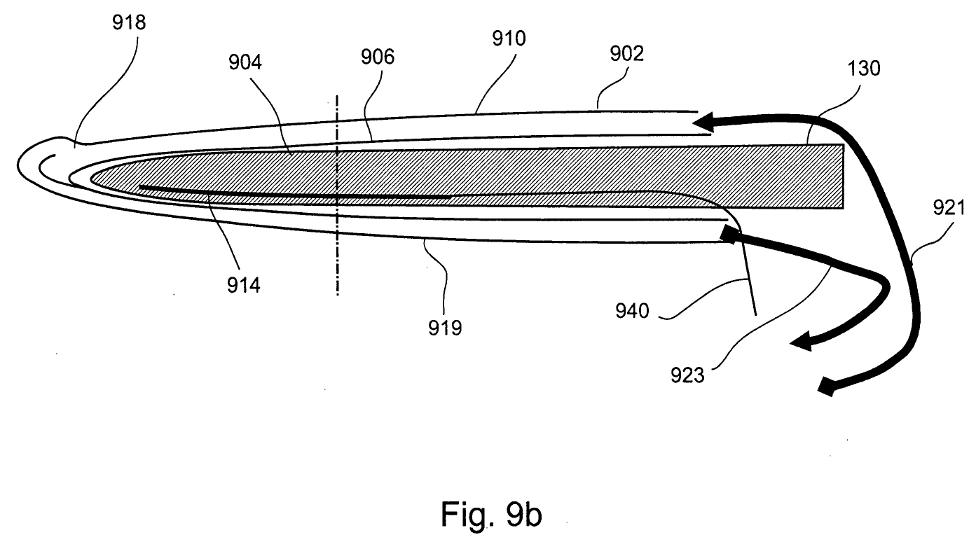

Attention is now drawn to FIGS. 9a and 9b, which are simplified schematics of a conductive liquid rectal warmer, according to an embodiment of the present invention.

FIG. 9a presents a cross sectional view and FIG. 9b presents a side view of a conductive liquid rectal warmer sleeve 910. A dashed line in FIG. 9b shows the position of the cross section shown in detail in FIG. 9a.

Conductive liquid rectal warmer sleeve 910 is presented with a rectal ultrasonic probe 130 inserted therein.

Sleeve 910 comprises a conductive liquid compartment 904, filled with electrically conductive liquid such as saline. Voltage applied between the two exposed electrodes 914a and 914b inside conductive liquid compartment 904 causes an electric current to flow between electrodes 914, producing heat and warming the liquid. Preferably, sleeve 910 is so constructed that current flows across portions of the device which are adjacent to the rectal wall to be warmed.

In similarity to embodiments previously discussed, insulated electric wires 940 for supplying voltage to exposed electrodes 914 are connected to a controller 840 (not shown in FIG. 9). Thermal sensors 860 (not shown in FIG. 9) may be installed in or near sleeve 910 for monitoring and regulation of temperature.

Depending on the conductive liquid used, electric current flowing therethrough may produce gas bubbles that interfere with ultrasonic imaging. Appropriate selection of conductive liquid and/or appropriate voltage settings may reduce or eliminate bubble creation. Alternatively, a bubble venting conduit 919 connected to conductive liquid compartment 904 at bubble venting orifice 918 may be used for venting gas produced by disassociation of components of the conductive liquid. Optionally, flexible liquid outlet hose 923 is used for removing bubble-liquid mixture from the vicinity, while flexible liquid inlet hose 921 replenishes the liquid level in sleeve 910. In this case, the patient is preferably positioned on his side, so that bubble venting orifice 918 may be positioned at a high point of sleeve 910, which high point will not be positioned between sleeve 910 and the patient's prostate.

Hoses 921 and 923 may be connected to a circulating pump equipped with means for removing bubbles from the liquid. Alternatively, an open loop of liquid flow may be used. For example, saline drip, in common medical use, may be connected to inlet hose 921, and liquid exiting from outlet hose 923 may be allowed to drain or be collected in a suitable receptor, such as an empty infusion bag.

As noted in the background section hereinabove, many surgeons will find it preferable or essential to be able to move an ultrasound probe freely within a rectum while monitoring cryoablation procedures. It is hence important to provide embodiments of rectal protection devices which explicitly enable free motion of ultrasound probes while continuing to protect rectal tissues. The embodiment presented by FIG. 3d and discussed hereinabove presents such a capability. Several additional embodiments providing this capability are now presented.

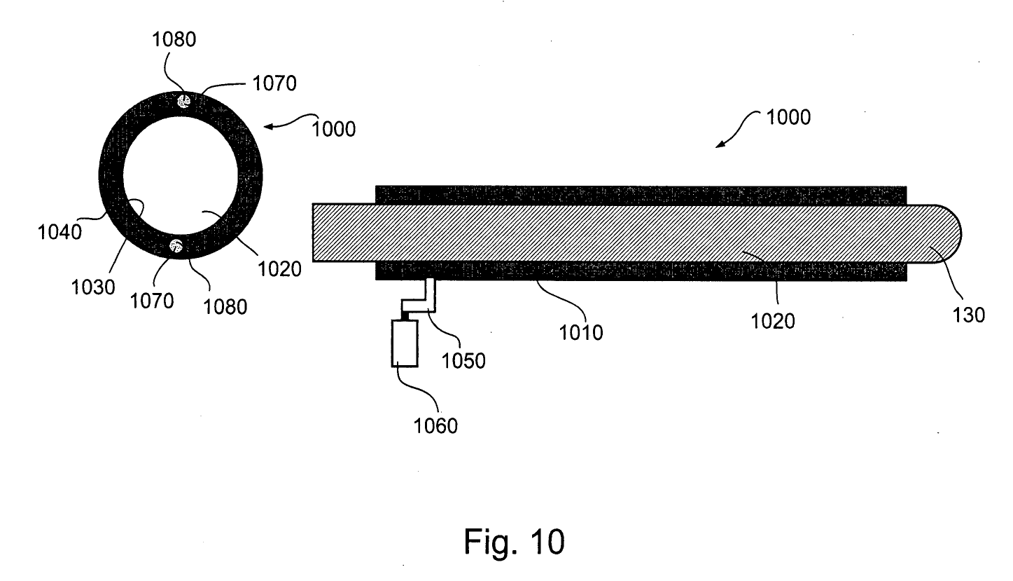

Attention is now drawn to FIG. 10, which is a highly simplified schematic of a rectal protection device designed to facilitate cryoablation of prostate tissues while protecting tissues of a rectum, enabling gentle heating of rectal tissues during ultrasound viewing, and enabling to moving an ultrasound probe freely within the rectum without compromising the heating process.

FIG. 10 presents lateral and cross-sectional images of a rectal protection device 1000 designed to enable free movement of an ultrasound probe within a rectum without compromising ultrasound viewing of a prostate and without compromising protective heating of rectum tissues;

Device 1000 comprises a flexible shaped sleeve 1010, constructed of a material which is which sufficiently solid to hold its form, yet sufficiently flexible to be a good transmitter of ultrasound vibrations. Soft silicon rubber or stiffened ultrasound gel are appropriate materials for sleeve 1010. Sleeve 1010 may comprise only such gel-like material, or alternatively may comprise a soft gel-like substance contained within thin and flexible walls of silicon, rubber or a thin flexible plastic.

Sleeve 1010 is thus substantially transparent to ultrasound waves, and an ultrasound probe contained therein can send and receive ultrasound waves propagated through sleeve 1010 into body tissues around sleeve 1010.

Sleeve 1010 is sufficiently stiff to be inserted into a rectum together with an ultrasound probe inserted therein, and may have a thinned distal edge to facilitate such insertion. Internal lumen 1020 of sleeve 1010 is sized to accommodate an ultrasound probe 130. Once sleeve and probe are inserted, probe 130 is free to be advanced and retracted within sleeve 1010, enabling ultrasound viewing from any point within the rectum, without requiring further movement of sleeve 1010 within the rectum. To facilitate movement of probe 130 within sleeve 1010 and to facilitate maintenance of position of sleeve 1010 within a rectum, inner wall 1030 defining lumen 1020 is preferably constructed as a smooth and low-friction surface, while outer wall 1040 of sleeve 1040 may be surfaced in such a manner as to provide friction or suction to gently impede motion of sleeve 1010 within a rectum. Alternatively, a handle 1050 attachable to a position-fixing fixture 1060 may be provided, for assuring that sleeve 1010, once installed in a rectum, will be substantially immobile when probe 130 is moved longitudinally within the rectum or even completely withdrawn therefrom. Of course, such immobilization is neither powerful nor permanent: when sleeve 1010 is no longer needed, it can simply be pulled from the rectum by a surgeon.

Sleeve 1010 preferably comprises a heating element 1070. Heating element 1070 may comprise electrical resistance wires embedded in a gel or similar substance. Alternatively, heating element 1070 may comprise a strip of conductive rubber operable to be used as an electrical resistance heating element. Conductive rubber has advantage of being relatively transparent to ultrasound vibrations, so as to not to impede functionality of the ultrasound probe. For simplicity, electrical wires appropriate for connecting element 1070 to a power source and/or a heating controller such a controller 640 are not shown in the Figure, but presence of such wires and use of such sensors and controllers is to be understood. Use of thermal sensors and controllers for controlling electrical heating elements has been discussed in the context of various embodiments described hereinabove, and those discussions are to be understood to apply as well to device 1000 and to other devices utilizing electrical heating presented hereinbelow.

Alternatively, heating elements 1070 may be conduits 1080 for a circulating fluid, such as a warmed saline solution. In similarity to various embodiments presented hereinabove, conduits 1080 are to be understood to be connectable to pump and heating systems such as those discussed with reference to FIGS. 2, 3a and 3b. Elements 1070 may also be used to circulate a cool fluid such as cool saline solution, allowing sleeve 1010 to be used for cooling as well as heating.

Elements 1070 are disposed within sleeve 1010 in a configuration appropriate to provide sufficient heat (or cooling) coverage to protect tissues, yet which avoids substantial interference with ultrasound viewing. Thus if, as is preferable, conductive rubber or soft conduits filled with liquid are used, ultrasound viewing directly through these elements is possible. If electrical resistance wiring is used, such wiring must be disposed in a pattern which minimizes obscuring of transmission to and from ultrasound transceivers of probe 130, and markings must be provided on sleeve 1010 enabling to rotationally orient sleeve 1010 with respect to probe 130 so that those ultrasound transceivers remain unobscured when probe 130 is inserted in sleeve 1010.

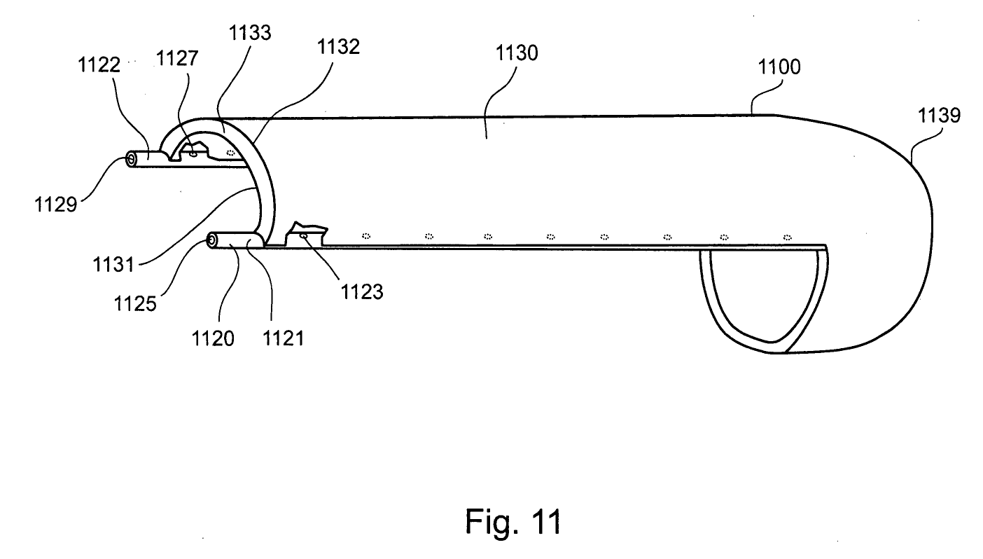

Attention is now drawn to FIGS. 11-13, which are simplified schematics of a rectal protection device 1100.

FIG. 11 presents device 1100 which comprises a semi-rigid frame 1120 having branches 1121 and 1122 joined by a flexible pouch 1130, according to an embodiment of the present invention.

Pouch 1130 is formed of either one or two layers of a material such as silicon rubber, latex, or plastic. Material used should be flexible but not necessarily expandable. (A cloth such as that used to fabricate umbrellas could be used, for example.) Device 1100 is designed so as to be insertable into a rectum together with an ultrasound probe 130, yet is independent of probe 130. Though device 1100 and probe 130 will touch when inserted together into a rectum, they are not physically connected one to another. Thus, presence of device 1100 does not impede free movement of probe 130 with respect to rectum and prostate. Probe 130 may move forward and backward in a rectum while device 1100 remains immobile.

Frame 1120 and pouch 1130 together provide an ultrasound-transparent means for heating that portion of a rectal wall situated between probe 130 and a prostate area. Heating of pouch 1130 may be accomplished by electrical heating or by flow of heated fluid, in similarity to embodiments presented hereinabove. In particular, pouch 1130 may be embodied with an inner layer 1131 and an outer layer 1132, layers 1131 and 1132 defining a volume 1133 (preferably of fixed volume). A heated (or cooled) fluid is provided by a heating/pumping apparatus such as those presented by FIGS. 2 and 3. Heated fluid flows through a conduit 1125 in frame 1121, passes through perforations 1123 into volume 1133, flows across pouch 1130, is received through perforations 1127 and passes into a conduit 1129 within frame 1122, whence it is exhausted, to be discarded or to be returned to a recirculating pump in a closed-loop system.

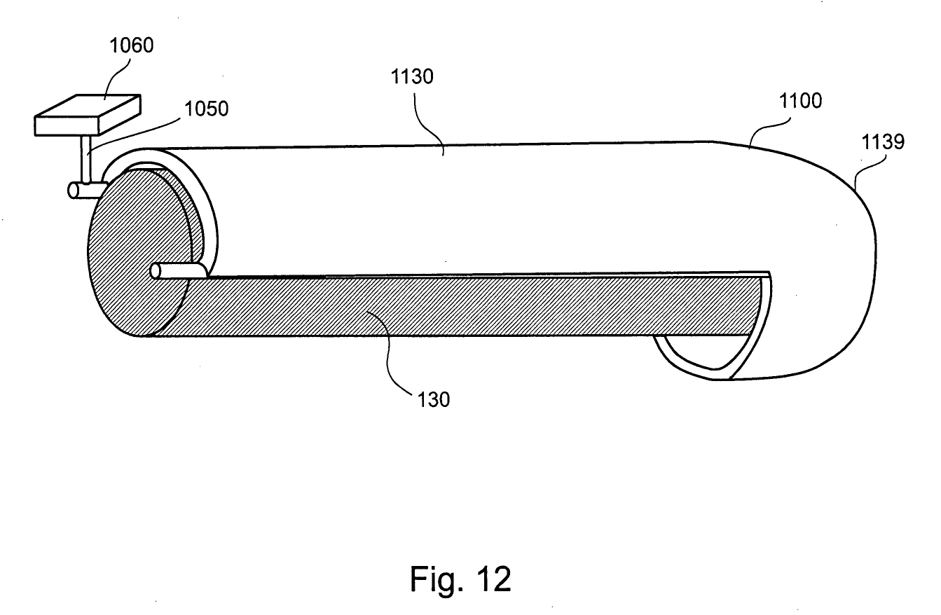

FIG. 12 presents an additional view of device 1100, showing positioning of pouch 1130 with respect to an ultrasound probe 130, which probe is free to move longitudinally within a rectum without thereby displacing device 1100. Optional handle 1050 attachable to a position-fixing fixture 1060 may be provided to fix position of device 1100 when device 1100 is inserted in a rectum, thereby facilitating moving probe 130 without displacing device 1100.

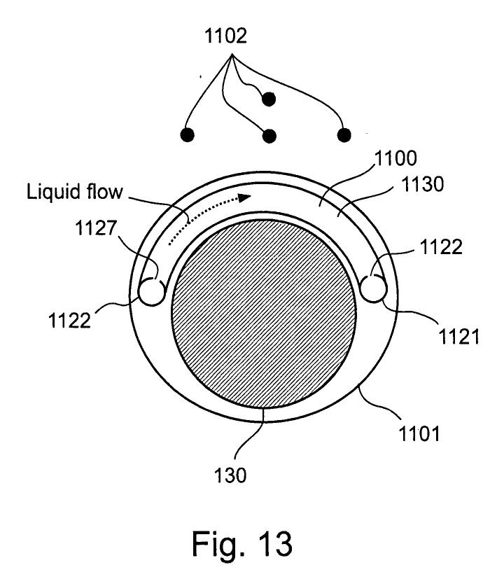

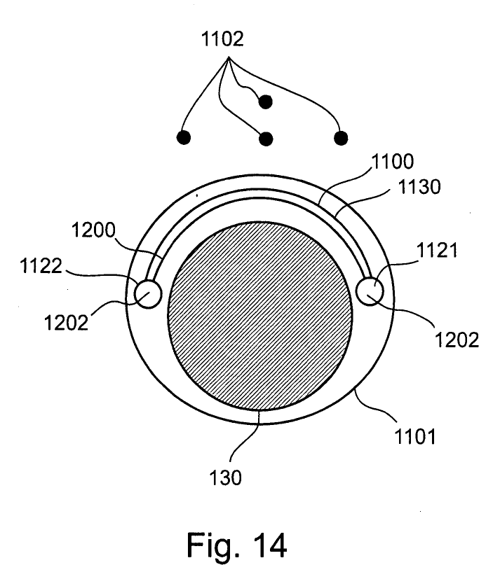

FIG. 13 presents a cross-sectional view of device 1100 within a rectum 1101 showing fluid flow within pouch 1130. From FIG. 13 it is clear that ultrasonic waves may be used to image the zone of interest, for example a tissue undergoing cryoablation by cryoprobes 1102, without being obstructed by frame members 1121 and 1122.

Attention is now drawn to FIG. 14, which is a simplified schematic of an alternative embodiment of rectal protection device 1100 using electrical heating, according to an embodiment of the present invention.

As presented in FIG. 14, pouch 1130 comprises an electrical heating element 1200, which is preferably a conductive rubber heating element. Element 1200 is connected through wires contained in frames 1121 and 1122 to a source of electric power controlled by a controller such as controller 640. Thermal sensors (not shown in the Figure) are preferably used to provide feedback to controller 640, enabling maintenance of heating element 1200 and of rectal tissues near device 1100 at appropriate temperatures, as discussed hereinabove.

In a further alternative construction, fine electrical resistance wires embedded in rubber, plastic, or a gel solution may be used in place of a conductive rubber strip as heating element 1200. Such wires would extend from frame 1121 to frame 1122 and would connect through wires running through those frames to electrical power source and control elements.

In a preferred embodiment shown in FIGS. 11 and 12, a distal end of pouch 1130 is formed as a ‘pocket’ shape 1139. Pocket 1139 is sized to accommodate a distal end of an ultrasound probe, and is provided to facilitate insertion of pouch 1130 into a body cavity. In a recommended method of use, an ultrasound probe 130 is coated with ultrasound gel and positioned with its distal end in pocket 1139. Pushing probe 130 into a rectum or other body cavity thus results in pushing device 1100 into that body cavity along with probe 130. Preferably device 1100 is pushed into (e.g.) the rectum as far as possible, whereupon device 1100 remains deeply inserted and probe 130 is free to move backwards and forwards in the rectum to varied viewing positions at the convenience of the surgeon.

In further alternate configuration, heating pouch 1130 can extend to cover more or less of the surface of probe 130. For proper functioning, given a patient in typical position for prostate cryoablation, heating of tissues above probe 130 is essential, whereas heating of tissues beside and below probe 130 is likely to be unnecessary.

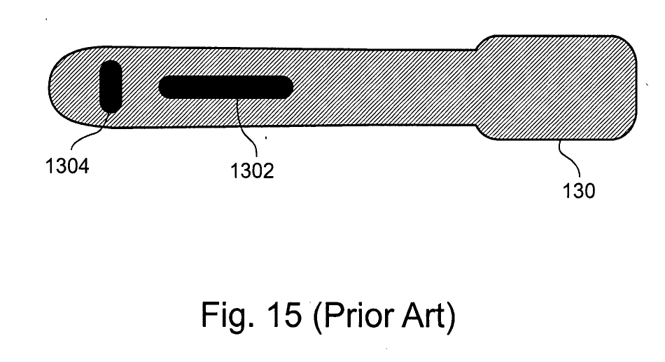

Attention is now drawn to FIGS. 15-17, which are simplified schematics presenting an ultrasound probe with internal heating element, according to an embodiment of the present invention. It is noted that despite the advantages explained hereinabove with respect to having a tissue protecting device which is physically independent from an ultrasound imaging device, under certain conditions a surgeon may prefer to use a coordinated device, if such a device provides significant advantages of convenience of operation. Accordingly, an ultrasound probe 1300 incorporating a heating element is here provided.

FIG. 15 presents an ultrasound probe 130 according to methods of prior art, showing typical positioning of an axial ultrasonic transceiver 1302 and a transverse ultrasonic transceiver 1304 on that probe. Heating elements provided on or within an ultrasound probe must avoid obscuring operation of those two transceivers; other portions of probe 130 may be provided with ultrasonically opaque electrical or other heating elements without thereby impeding ultrasound imaging operation of the probe.

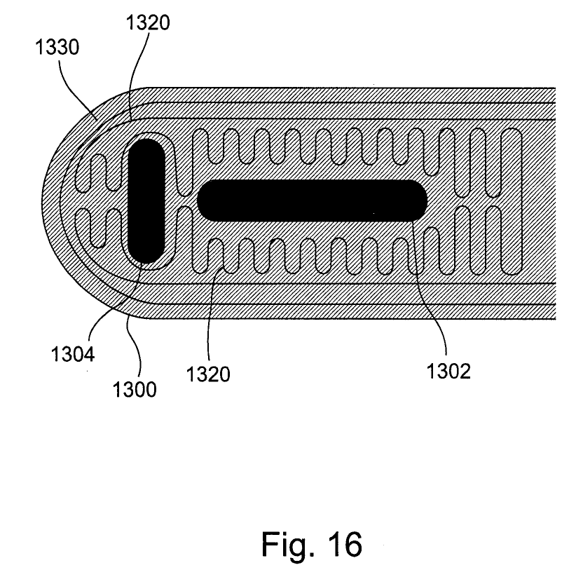

Attention is now drawn to FIG. 16, which is a simplified schematic of an ultrasound probe 1300 which incorporates an electric resistance heating element 1320, according to an embodiment of the present invention. As may be seen from FIG. 16, resistance heating element 1320 is disposed on or under the surface of probe 1300 in such a manner as to provide relatively even distribution of heat over at least a portion of that surface. (Distribution of heat across that surface which faces in the direction of the field of view of probe 1300 is essential, since that is the direction expected to be cooled by the cryoablation process. Distribution of heat on other sides of probe 1300 is optional. For convenience of exposition, that surface of probe 1300 which faces in the direction of the field of view of probe 1300 will be referred to as the “upper face” of probe 1300 in the following.)

At least an upper face of probe 1300 is preferably coated with a heat diffusion layer 1330. Heat diffusion layer 1130 is preferably constructed of a material which is relatively transparent to ultrasound waves, and which has a relatively high coefficient of heat conduction. Ultrasound-transparent materials are not typically good heat conductors, but perfect heat conduction is not needed; all that is required is a level of heat conduction which ensures sufficient distribution of heat to portions of the rectal tissue which are directly above transceivers 1302 and 1304. Such a level of heat conduction will serve also to provide smooth distribution of heat generated by heating elements 1320 over probe surfaces adjacent to elements 1320, thereby providing an ultrasound probe surface with a fairly even distribution of heat, without dramatic hot spots or cold spots. A gel material may be used to provide such a layer, or alternatively a closed liquid pocket encased between two layers of latex might be used, or any similar set of materials could be provided. Diffusion layer 1330 can serve not only to enhance heat distribution, but also to enhance ultrasound wave transmission by distancing the wave transmitting transceivers of probe 1300 from the rectal tissues, as may be required for good ultrasound viewing.

In an alternate construction, conductive rubber elements may be used in place of resistive wire, to further enhance heat distribution and to minimize interference with transmission and receipt of ultrasound waves.

Of course, in this embodiment as in other electrical heating embodiments presented above, thermal sensors on probe 1300 and/or in rectal tissues are preferably provided, and feedback from those thermal sensors is preferably directed to a controller 640 which supplies power for heating elements 1320 of probe 1300.

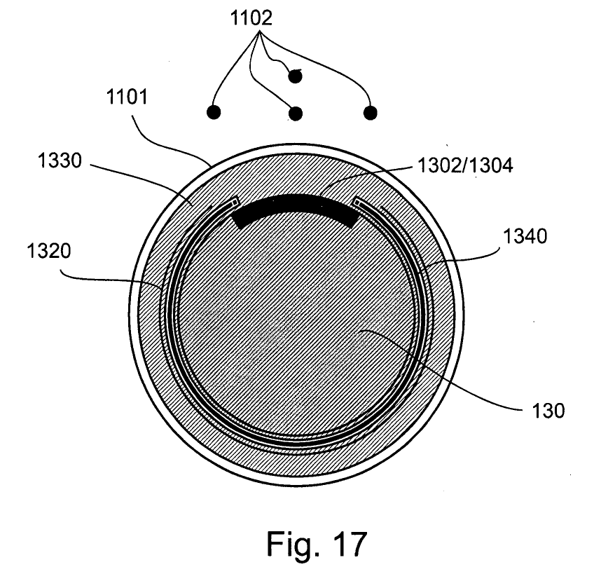

Attention is now directed to FIG. 17, which is a simplified schematic providing a cross-sectional view of probe 1300, according to an embodiment of the present invention. As seen in FIG. 17, a thermal insulation element 1340 may optionally be provided to isolate heating elements 1320 from transceivers 1302 and 1304 and from other components of probe 1300 which are liable to be damaged by exposure to excessive heat. At the surface where layer 1330 comes in contact with rectal tissue, a desirable temperature would be in the vicinity of 42° C. Layer 1330 is preferably at least a few millimeters thick, so as to provide volume for sufficient diffusion of heat from heating elements beside transceivers 1302/1304 into layer 1330 surfaces above transceivers 1302/1304. Since materials providing ultrasound transparency are not excellent heat conductors, temperatures at heaters 1320 will need to be somewhere in a range of approximately 60°-90° C. Such high temperatures may tend to damage delicate ultrasound components. To prevent such damage, thermal insulation element 1340 is provided to thermally insulate delicate internal components of probe 1300 from heater 1320.

It is noted that when probe 1300 is used for the specific clinical purpose of protecting the rectal wall during cryoablation of prostate tissue (and for various other specific purposes), heating of tissues may need to be provided only on one side of the probe, since only one side of the probe will be facing areas cooled by the cryoablation procedure. Consequently, depending on intended uses of a specific implementation of probe 1300, heating element 1320 and heat diffusion layer 1330 may be provided on all surfaces of probe 1300, only on the upper face of probe 1300, or on any other combination which serves the purposes of a surgeon in a particular clinical context.

The foregoing discussion has presented probe 1300 as a single unit, with heating element 1320, diffusion layer 1330, and optional insulation layer 1340 integrated into probe 1300 as a single re-useable unit. It is noted that in an alternative construction elements 1320, 1330 and 1340 may be presented in the form of an independent sleeve such as that presented in FIG. 10 and discussed hereinabove. In such an embodiment, insulation layer 1340 will of course be positioned as an interior layer of sleeve 1010, heating elements 1320 will be positioned as an intermediate layer of sleeve 1010 exterior to insulation layer 1340, and heat diffusion layer 1330 will be positioned as an external layer of sleeve 1010 exterior to heating elements 1320. Heating elements 1320 (also labeled elements 1070 of sleeve 1010 of FIG. 10) would then be laterally positioned in a manner similar to that shown in FIG. 16, leaving appropriately-sized ‘windows’ for transceivers 1302 and 1304.

Such a sleeve 1010 could then be pulled over a conventional cryoprobe 130 to produce a functional equivalent of probe 1300, having the advantage that sleeve 1010, including its heating elements 1070, may be presented in a form suitable for sterile one-time disposable use. As noted, in such an embodiment, external markings or embedded transparencies should be provided on sleeve 1010 and preferably also on probe 130, to facilitate accurate placement of sleeve 1010 on probe 130 in a manner which avoids obscuring transceivers 1302 and 1304 of probe 130. Optionally, ultrasonically transparent windows are also optically transparent so that the user can verify proper alignment of the windows over the ultrasonic transducers.

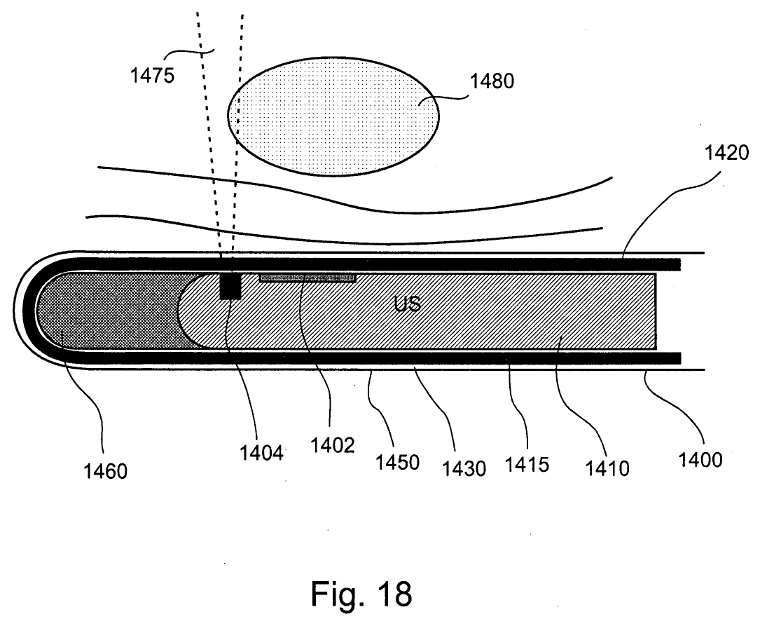

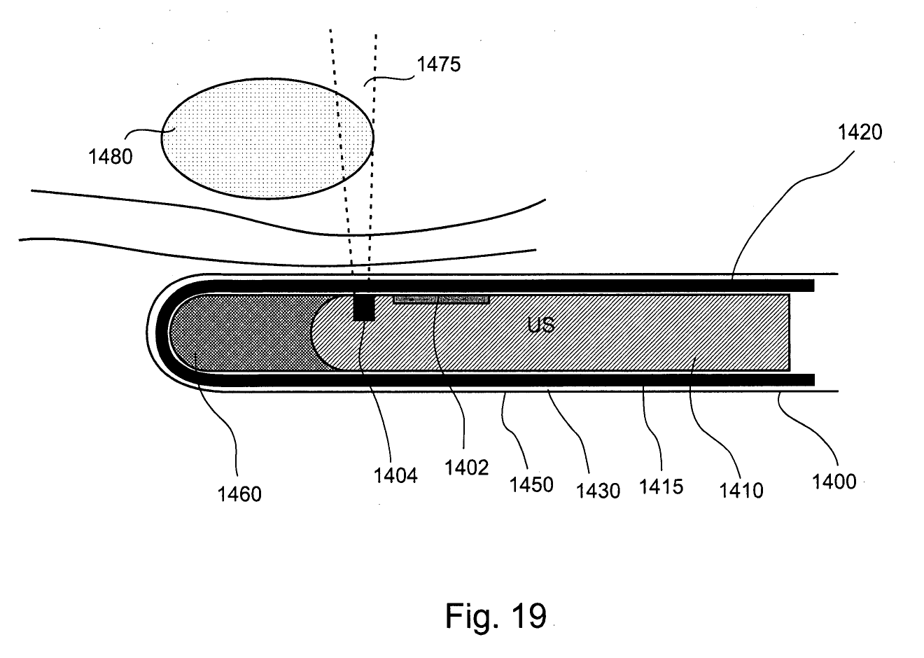

Attention is now drawn to FIGS. 18 and 19, which are simplified schematics of a device 1400 comprising an ultrasound probe and heater, and having an extended distal portion, according to an embodiment of the present invention. FIGS. 18 and 19 show two a same device inserted at two different positions in a rectum.

Considering first FIG. 18, the FIG. 18 presents a heater-ultrasound probe combination labeled device 1400. Device 1400 represents an additional solution to the problem described in the background section hereinabove, that preferred clinical practice requires that a surgeon, in order to view all areas of the prostate, must be able to freely move a rectal ultrasound probe, yet prior-art ultrasound probe and heater combinations are such that partial withdrawal of the ultrasound probe, required to view near regions of the surgical target area, result in exposure of far regions of the rectal wall to danger of freezing during cryosurgery (or of excessive heating during heat-ablative surgery).

Device 1400 comprises an ultrasound probe 1410 having an axial ultrasonic transceiver 1402 and a transverse ultrasonic transceiver 1404, a layer 1420 comprising heating elements as variously described hereinabove, an optional heat diffusion layer 1430 similar to layer 1330 described above. In use device 1400 is optionally covered by a condom 1450 or other rubber or latex outer wall. In other words, device 1400 can comprise an ultrasound probe comprising a heater, as presented by FIG. 16, or alternatively device 1400 can be an ultrasound probe 1410 without heating element, combined with a sock-like heating sheath 1415 which comprises heating element 1420 and an optional heat diffusion (e.g. gel) layer 1430. Heating element 1420 may be a wire resistance heater, a conductive rubber resistance heater, any of the fluid-flow heating systems described above, or any other appropriate heater, or may be a fluid-flow mechanism for cooling.

Whether device 1400 is constructed as a single unit or as an independent ultrasound probe covered by sheath 1415, device 1400 is characterized in that transceivers 1402 and 1404 are distanced from a distal end of device 1400 by at least 3-4 cm, and heating element 1420 extends at least 2 cm and preferable 2-4 cm beyond the most distal of transceivers 1402 and 1404. Device 1400 is designed so that sheath 1415, if independent from probe 1410, moves together with probe 1410 when probe 11410 is encased in sheath 1415 and the two are inserted in a rectum.

Several alternative methods of construction are suggested. Sheath 1415 and probe 1410 may be constructed as a single unit, with transceivers 1402 and 1404 distanced from the distal end of that unit as described above. In a first alternative construction, probe 1410 may be a standard (i.e. prior art) ultrasound probe. In this case a passive extender 1460, constructed of plastic or other appropriate material, and preferably shaped to conform to the shape of a specific model of ultrasound probe 1410, may be inserted into sheath 1415 prior to insertion of probe 1410 into sheath 1415. In a second alternative construction sheath 1415 may be supplied with extender 1460 already installed at its distal portion.

Thus, in all of the above-described combinations, the user is presented with a device 1400 which comprises a heating element extending to its distal end or nearly to its distal end, and ultrasound transceivers distanced from that distal end, preferably by 2-4 cm or more. Device 1400, so constituted, is operable to heat all portions of the rectum which are near the prostate while probe 1410 is utilized to image the prostate region during cryosurgery. In particular, without interrupting heating of endangered rectal tissues, device 1400 may be moved freely forwards and backwards in a rectum sufficiently to enable the surgeon to image near regions of the prostate target area, yet far regions of rectal wall will continue to be protected from freezing. FIG. 18 shows device 1400 so positioned that transceiver 1404 has a field of view (labeled 1475) which enables imaging of the far side of prostate 1480. In comparison, FIG. 19 shows device 1400 somewhat retracted in the rectum, so that field of view 1475 enables imaging the near edge of prostate 1480. As may be seen from inspection of FIG. 19, portions of rectal wall which are in close proximity of prostate 1480 are protected by heater 1420 in both positions of device 1200, in contrast to prior art devices which would successfully protect rectal tissues at the field-of-view position shown in FIG. 18, but would endanger portions of rectal tissue at the field-of-view position shown in FIG. 19.

Attention is now drawn to an innovative use for various devices presented hereinabove. Descriptions herein of exemplary uses of embodiments of the present invention have emphasized use of heating of body conduit tissues such as rectal tissues to protect them from damage during intense cooling caused by cryoablation in nearby organs, and to use of cooling of body conduit tissues such as rectal tissues to protect them from damage during intense heating caused by heat-producing ablation procedures in nearby organs. It has been also been emphasized that control of thermal conditioning processes (heating and cooling) within devices of the present invention is preferably accomplished utilizing a controller (e.g. controllers 230, 340, 640, 730, 840) receiving thermal information from thermal sensors (e.g. 234, 360, 631, 734, 860) to control heating and cooling by devices here presented, control being based on temperature information received from sensors. It is now noted that use of a heating device (such as those presented hereinabove, or other heating devices) at a first position, during cryoablation of nearby tissues at a second position, enables fine control of the extent of cryoablation destruction zones and fine control of the extent of freezing surrounding cryoablated tissue. For example, utilizing feedback from thermal sensors, or using treatment protocols established through prior clinical experience or through experimentation, it is possible, by controlling temperature of one or more cryoprobes on the one hand and temperature of one or more heating devices on the other hand, to cause formation of an isotherm at a desired temperature at or near a selected position within body tissue. Thus, for example, control of temperature of a rectal heater on the one hand and of a set of cryoprobes on the other hand enables not only to protect rectal tissue in general, but to determine in advance, and with a fair degree of accuracy, just how close to a rectal wall freezing of tissues will be allowed to progress. Appropriate balancing of heating within a urethra and cooling within a prostate allows to determine with a fair degree of accuracy how large a border of undestroyed prostate tissue will be allowed to remain around that urethra during prostate cryoablation. In addition to information from thermal sensors in cryoprobes, in heaters, and in body tissue, feedback from other sensors (e.g. pressure sensors operable to detect freezing of tissues) and from visualization modalities such as MRI and ultrasound can be used to affect control of cooling processes on the one hand and a heating process on the other hand. Such control can be accomplished manually by a surgeon, or automatically by a processor such as controllers 230, 340, 640, 730, 840, operating according to stored algorithms, or by a combination of both manual and automatic control.

It is further noted that an additional advantage of combining heating at a first position with cooling at a second position, as here described, is that such a process reduces substantially the volume of damaged tissue between ablated tissue and undamaged tissue. Under cryoablation without simultaneous heating, a volume of complete tissue destruction is always surrounded by an even large volume within which tissue functionality is not complete destroyed, but is damaged. It is clearly a desirable goal in cryosurgery to reduce the amount of such damaged tissue. Utilizing heating devices, such as those described hereinabove, in proximity to cooling devices such as cryoprobes, enables to create a sharp temperature gradient between cold source and heat source, and thereby significantly reduce the volume of damaged tissue surrounding destroyed tissue of a cryoablation target.

It should be appreciated that although the invention presented hereinabove has been primarily described with reference to the illustrative example of protection of rectal walls during cryoablation of the prostate, this illustration is not intended to be limiting. The embodiments described, with minor and obvious alterations, may be used to protect tissues in various other surgical contexts For example, the embodiments described can be used to protect tissues in body cavities other than the rectum, and embodiments comprising fluid flow elements may be used to protect tissues from heat damage as well as protecting tissues from cold damage.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A device for facilitating ablation of tissues near a body cavity, comprising a flexible thermal conditioner insertable together with an ultrasound probe into said body cavity, wherein
(a) at least a portion of said thermal conditioner comprises ultrasound-transmissive material operable to transmit ultrasound waves between said ultrasound probe and body tissues external to said body cavity when said thermal conditioner and said ultrasound probe are together inserted into said body cavity and said ultrasound probe is operated to image tissues; and
(b) said thermal conditioner is designed as to allow said ultrasound probe to move freely relative to said conditioner when said conditioner and said ultrasound probe are together inserted in said body cavity.

2. The device of claim 1, wherein said thermal conditioner is formed as a sleeve having a lumen sized to accommodate an ultrasound probe.

3. The device of claim 1, wherein said thermal conditioner is shaped as a pouch having a distal pocket sized to accommodate a distal end of an ultrasound probe.

4. The device of claim 1, wherein said thermal conditioner further comprises a conductive rubber electrical resistance heater.

5. The device of claim 1, further comprising an immobilizer operable to immobilize said thermal conditioner with respect to said body cavity when said thermal conditioner is inserted in said body cavity.

6. A device for facilitating ablation of tissues near a body cavity, comprising a flexible sleeve insertable in said body cavity, said device comprises an internal lumen sized to accommodate an ultrasound probe inserted in said sleeve, wherein
(a) at least a portion of said sleeve comprises ultrasound-transmissive material operable to transmit ultrasound energy between an ultrasound probe inserted in said sleeve and body tissues external to sleeve; and
(b) said internal lumen is so sized and surfaced as to allow an ultrasound probe inserted therein to move freely within said sleeve when said sleeve is inserted in said body cavity.

7. The device of claim 6, wherein said sleeve is designed and constructed to maintain thermal contact with a rectal wall when said sleeve is inserted in a rectum and an ultrasonic probe initially inserted in said sleeve is withdrawn therefrom.

8. The device of claim 6, wherein said sleeve further comprises a thermal conditioning element.

9. The device of claim 8, wherein said thermal conditioning element is a heating element.

10. The device of claim 8, wherein said thermal conditioning element is a cooling element.

11. The device of claim 9, wherein said heating element is an electrical resistance heater.

12. The device of claim 11, wherein said electrical resistance heater comprises conductive rubber operable to heat when traversed by an electric current.

13. The device of claim 11, wherein said electrical resistance heater comprises electrical resistance wires encased in a flexible, ultrasound-transparent material.

14. The device of claim 6, further comprising a conduit operable to conduct a fluid through at least a portion of said sleeve.

15. The device of claim 14, further comprising a pump for pumping a fluid through said conduit.

16. The device of claim 15, wherein said pump is a peristaltic pump.

17. The device of claim 15, wherein said conduit is formed as a closed-circuit conduit.

18. The device of claim 15, wherein said pump is a peristaltic pump and said conduit is formed as a closed-circuit conduit.

19. The device of claim 14, further comprising a heater operable to heat a fluid flowing through said conduit.

20. The device of claim 14, further comprising a cooler operable to cool a fluid flowing through said conduit.

21. The device of claim 6 wherein said sleeve comprises a gel operable to transmit ultrasound waves.

22. The device of claim 6, sized to be insertable into a rectum.

23. The device of claim 6, so shaped and configured that at least one model of ultrasound probe may be so inserted in said sleeve that when said sleeve is inserted in a body cavity ultrasound transceivers of said inserted ultrasound probe are operable to send and receive ultrasonic waves through said sleeve.

24. The device of claim 8, further comprising thermal insulation, said thermal insulation being so positioned that when an ultrasonic probe is inserted in said sleeve, said thermal insulation at least partially insulates said inserted ultrasonic probe from said thermal conditioning element.

25. The device of claim 6, further comprising an immobilizer operable to immobilize said device with respect to a body cavity into which said device is inserted, thereby enabling an ultrasound probe inserted in said device when said device is inserted in a body cavity to be advanced and retracted within said device without thereby causing substantial displacement of said device within said body cavity.

26. A tissue protection device comprising a pouch having:
(a) a distal end formed as pocket sized to accommodate a distal end of an ultrasound probe; and
(b) an inner sheath wall and an outer sheath wall, said inner sheath wall and outer sheath wall defining a volume, wherein said volume comprises a semi-rigid sound-transmitting material.

27. The device of claim 26, wherein said ultrasound-transmitting material is a gel.

28. The device of claim 26, wherein said volume further comprises a conduit for conducting a fluid through at least a portion of said volume.

29. The device of claim 28, further comprising a frame which comprises a fluid input lumen communicating with said conduit and a fluid exhaust lumen communicating with said conduit.

30. The device of claim 28, further comprising a pump operable to pump a fluid through said conduit.

31. The device of claim 30, further comprising a heater operable to heat said fluid.

32. The device of claim 30, further comprising a cooler operable to cool said fluid.

33. The device of claim 30, wherein said pump is a peristaltic pump.

34. The device of claim 30, wherein said conduit forms a closed circuit.

35. The device of claim 26, wherein said volume further comprises an electrical heating element.

36. The device of claim 35, wherein said electrical heating element comprises electrical resistance wires embedded in said material.

37. The device of claim 35, wherein said electrical heating element comprises conductive rubber.

38. The device of claim 26, further comprising an immobilizer operable to immobilize said pouch with respect to a body cavity into which said pouch is inserted, thereby enabling an ultrasound probe inserted in said pouch when said pouch is inserted in a body cavity to be advanced and retracted within said pouch without thereby causing substantial displacement of said pouch within said body cavity.

39. The device of claim 26, wherein an inner surface of said inner pouch wall is so designed and constructed that an appropriately lubricated ultrasound probe inserted in said pouch is enabled to slide easily forward and backward within said pouch, and an exterior surface of said outer pouch wall is designed and constructed to impede easy movement of said pouch with respect to walls of a body cavity when said pouch is inserted in said body cavity, said construction enabling an ultrasound probe inserted in said pouch when said pouch is inserted in a body cavity to be advanced and retracted within said pouch without causing substantial displacement of said pouch with respect to said body cavity.

40. A sheath for protecting a first tissue during ablation of a second tissue, which second tissue is distant from said first tissue, comprising:
(a) a first portion comprising material substantially transparent to ultrasound waves; and
(b) a thermal conditioning element operable to influence temperature of said first tissue;
wherein said first portion is so sized and shaped as to be operable to be positioned over ultrasound transceivers of an ultrasound probe when said ultrasound probe is inserted in said sheath.

41. The sheath of claim 40, wherein said thermal conditioning element is a heating element.

42. The sheath of claim 41, wherein said thermal conditioning element is a cooling element.

43. The sheath of claim 40, wherein said thermal conditioning element extends distally to a first position on said sheath, and said sheath further comprises a distal blocking element serving to allow insertion of an ultrasound probe only up to a second position in said sheath, and said second position is at least 2 cm proximal to said first position.

44. An insertion-blocking device insertable into a temperature-conditioning sheath sized to accommodate an ultrasound probe, said insertion-blocking device serving to distance a distal end of any such inserted probe from a distal end of any such sheath by at least 2 cm.

45. A system for protection of tissue of a body conduit during ablation of tissue near said body conduit, comprising:
(a) a sheath sized and shaped to fit over at least a portion of an ultrasound probe, said sheath being at least partially constructed of material transparent to ultrasound waves;
(b) a closed loop conduit operable to contain a fluid, a first portion of said conduit passing within a portion of said sheath and a second portion of said conduit being external to said sheath; and
(c) a peristaltic pump operable to be connected to said second portion of said conduit and to effect a flow in fluid contained within said conduit.

46. The system of claim 45, further comprising a heater operable to heat fluid within said conduit.

47. The system of claim 45, further comprising a cooler operable to cool fluid within said conduit.

48. The system of claim 45, wherein said closed loop conduit is hermetically sealed and a fluid is contained therein.

49. The system of claim 48, wherein said fluid is a liquid.

50. A sleeve for warming a body cavity during treatment of body tissues near said body cavity, said sleeve comprising:
(a) a lumen defined by an inner wall, said lumen being sized to accommodate an ultrasound probe;
(b) an outer wall surrounding said inner wall, said outer and inner walls together defining a volume;
(c) a fluid hermetically contained within said volume; and
(d) a heating element contained within said volume and operable to be positioned to one side of an ultrasound probe when said ultrasound probe is inserted in said sleeve and said sleeve is inserted in said body cavity.

51. A sleeve for heating a body cavity sized to accommodate an ultrasound probe insertable into said sleeve, comprising a vent in a distal portion of said sleeve, said vent serving to facilitate venting of air trapped between an ultrasound probe and said sleeve when said probe is inserted into said sleeve.
